(19) United States
(12) Patent Application Publication
(10) Pub. No.: US 2006/0111341 A1
(43) Pub. Date: May 25, 2006

(54) Use of galanthamine and the derivatives thereof in the production of medicaments

(76) Inventors: Angelika Bodenteich, Stevregg-Plesching (AT); Werner J Frantsits, Nothartgasse (AT); Eberhard Pirich, Mottlstrasse (AT); Laszlo Czollner, Ebenfurth (AT)

Correspondence Address: POPOVICH, WILES & O'CONNELL, PA;650 THIRD AVENUE SOUTH, SUITE 600, MINNEAPOLIS, MN, 55402, US

(21) Appl. No.: 10/537,568

(22) PCT Filed: Jul. 12, 2004
(86) PCT No.: PCT/AT04/00251

§ 371 Date: Jul. 15, 2005

(30) Foreign Application Priority Data
Sep. 29, 2003 (AT) A 1538/2003

(51) Int. Cl. A61K 31/55

(52) U.S. Cl. 514/215

(57) ABSTRACT

The invention relates to the use of galanthamine and the cholinergically active derivatives thereof in the production of medicaments for preventive treatment of postoperative delirium and/or subsyndronal postoperative delirium. Galanthamine, the galathamine derivative(4aS,6R,8aS)-6-hydroxy-3-methoxy-11-methyl-4a,5,9,10-tetrahydro-6H-benzofuro[3a,3,2-ef][2]benzazepinium bromide and analogous salts, hydrates or solvates are advantageously suited for use according to the invention.

The present invention relates to the use of galanthamine and galanthamine derivatives for manufacturing medicaments useful for the treatment of post-operative delirium.

Despite clear progress in the field of anesthesia as well as in the perioperative time, today a substantial portion of the patients, having large surgical procedures and interventions, suffer from post-operative psychiatric complications, more in particular this psychiatric complications fall broadly under the umbrella of post-operative delirium.

Delirium is a medical condition of disturbed consciousness, characterized by general confusion, reduction of cognitive functions (attention, concentration and memory), hallucinations and unstable emotions. Thus, delirium exhibits elements of dementia like other psychotic conditions, however it is distinguished from those conditions particularly by its acute nature and usually occurs spontaneously, even if often delayed and incomplete it is reversible.

Contrary to the degenerative dementia syndromes even when they are present and excluding functional disturbances of the central nervous system with post-operative delirium, the clinical picture produced by the individual psychiatric symptoms can fluctuate very fast—occasionally within seconds—.

Acute or subacute delirium (according to the medical classifications ICD 293.0 and/or 293.1 of the World Health Organisation) is often induced by intake or administration of pharmacologically effective substances. Numerous such substances are active substances or metabolites of medicines, so that a a medicament-induced delirium (ICD 292.81) can arise. In particular medicines with an anti-cholinergic effect, which partly block the nervous system based on the neurotransmitter acetyl choline, can induce a delirium, however sedatives, like benzodiazepines, and antimaniacals such as lithium salts can also induce delirium.

Also intoxicants and/or their acute withdrawal after chronic use can produce delirium. This occurs very frequently especially with acute alcohol abuse and/or in the case of alcohol withdrawal (ICD 291.0). However substances such as Cannabis, Amphetamine, cocaine etc. can also cause delirious conditions.

While the consciousness changes associated with a state of delirium mentioned above have a neurochemically directly comprehensible cause, there is also in the long run an unknown genesis for delirium. Also, despite the well-known techniques (surgical interventions) there is no doubt that one has to deal with post-operative delirium, since there may not be a basis for a pathological mechanism.

Post-operative delirium (POD) is regarded today as a multi-functional syndrome (1), whereby the age and the general state of health of the patient play a role, like possibly in preoperative existing cognitive disturbances, which may be influenced by the given defined anesthetic, and possibly also determined by intraoperative physiological changes (2). Although POD can occur immediately after awaking from the anesthetic provided, it is not to be equated with fast disorientation occurring after anaesthesia. Rather POD can also begin on the second post-operative day or also still later, after actual awakening or coming out from the given anesthesia after running its clinical course. Thus a direct effect of the perioperative given anesthestic and/or analgesics is to be excluded in these cases.

Although the scientific literature has contradictory data concerning the incidence of POD (which to a large extent points up to differences in the examined patient populations and the used psychiatric definition that is leading back to it), there exists nevertheless general agreement that it concerns a quite frequently arising phenomenon (3), in particular after large orthopedic surgical interventions (4) and particularly with older patients. A recently published study (5) found using the clinically very relevant and valid Confusion Assessment Method (CAM; 6) that of 2158 post-operative patients, 16% fully hinted at having delirium, 13% with at least two key symptoms, and 40% with at least one symptom, while only 32% were symptom-free.

Although POD arises thus frequently and almost exclusively with stationary or bedridden patients, and although it is considered as a bad prognostic indication to the further post-operative process, this condition is frequently not noticed or is not considered. This is to be attributed above all to the fact that post-operative patients usually remain under the supervision of the responsible surgical departments and that because of their apathy and stress (hypoactive) the personnel often do not recognize delirium. Only behavior-remarkable (hyperactive) patients are treated therapeutically with antipsychotics and/or sedatives (7). Already the therapy of the so-called subsyndromes of POD (which do not fulfill the psychometrics criteria of POD) would be extremely important, since its existence represents a risk factor for the progression and the time-frame of the delirious condition, and to what is statistically seen with an extended hospital stay, such as increased mortality after dismissal, and with later controlled investigations accompanies a decreased cognitive achievement (8); with the latter sequences one also speaks of Post-Operative Cognitive Decline (POCD), which condition can change into dementia.

The use of cholinesterase inhibitors for the therapy of medicament-induced delirium has been well-known for quite some time. This applies particularly to the “central anti-cholinergic syndrome” (9), however also to delirium, which does not arise in direct connection to treatments with directly anti-cholinergically working medicaments. The application of the prototypical cholinesterase inhibitor physostigmine is exemplarily mentioned with relevant complications not found with narcotically working acute sedatives (10).

The favourable experiences made thereby were transferred also to the POD. In 1978, in the literature, there were already recommendations for the avoidance of delirious conditions after completion of the anesthesia, by using the injection of a single dose physostigmine while under still the influence of the anesthetic (11). The therapy of an existing case, in particular one manifesting after a lucid post-operative period, does not address delirium itself however, so that this application must be rated as intraoperative prophylaxis of a substance-induced (directly with effects in connection with the anesthetic) delirium.

WO 00/032185 A reveals the effects on the cholinergic system to the therapy of delirium, and also under it the PODs, which is now called “cholinergic delirium”. In WO 00/032185 A, delirium is understood to develop within the succeeding 48 to 72 hours without a treatment or an infusion with substances which block the cholinergic system. WO 00/032185 A discloses the use of cholinesterase inhibitors for treating the PODs after an operation. Concrete examples of the use of galanthamine and its derivatives for treating PODs is disclosed in WO 00/32185 A. The WO 00/32185 A publication contains as the only example the case of a female patient, who had suffered a lithium intoxication and whose occurring delirium was successfully treated with the cholinesterase inhibitor “rivastigmine”, an irreversible inhibitor of the cholinesterases, which has its effect by covalent modification (carbamylation) in the course of the medicamentous therapy through many years of existing bipolar disturbance of these enzymes. The invention is concerned with medicament-induced delirium.

At present there are no suitable or accepted medicaments for the indication of POD as well as no published systematic clinical studies which support the specific effectiveness of a medicament with strict scientifically defined POD. Thus, there still exists a substantial medical need for a pharmacolocial means for treating fast occurring POD which terminates quickly. Special value must be put on minimum side effects of such a therapy, since a POD patient is by definition in the post-operative recovery phase and therefore exhibits reduced tolerance to physiological and psychological stress.

The invention is the basis to meet this need.

The use of galanthamine and galanthamine derivatives having cholinergic activity are the subject according to invention and their use for manufacturing medicaments for the treatment of post-operative delirium and/or subsyndromes of post-operative delirium.

Further the use of galanthamine and galanthamine derivatives having cholinergic activity are suggested according to the invention for manufacturing medicaments for the preventive treatment of post-operative delirium and/or subsyndromes of post-operative delirium.

Preferably the galanthamine derivatives have the general formula Ia 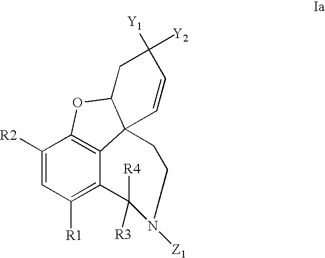
and the salts thereof, wherein R₁ is H, branched or straight chain (C₁-C₆) alkyl, Br, NO₂, NR₅R₆ wherein R₅ and R₆ are the same or different and are selected from H, branched or straight chain (C₁-C₆) alkyl, and wherein R₂ is OH, branched or straight chain (C₁-C₆) alkyl, methoxy, phenyloxy or the following group 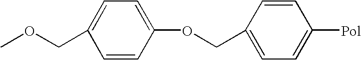
whereby Pol is a polymer, preferably one in accordance with WO-01/174820A1, and wherein R₃ and R₄ either at the same time or alternatively are H, D, CN, straight chain or branched (C₁-C₆) alkyl or a carbonyl group together, wherein Y₁ and Y₂ alternatively are H or a group selected from: 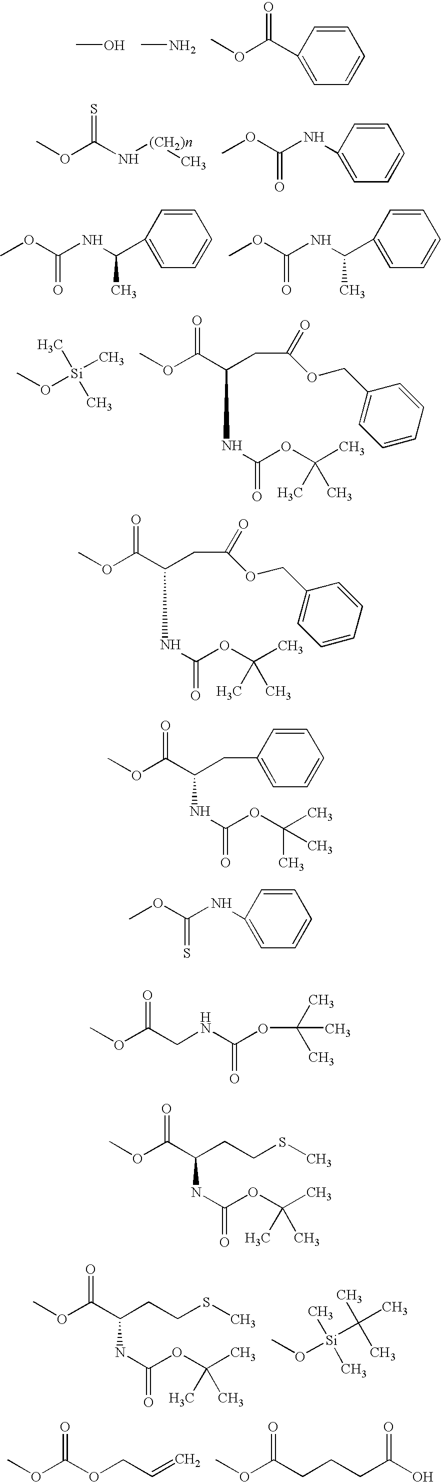 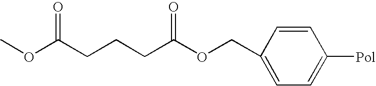
wherein n represents a value of 0, 1 to 15, and Pol has the meaning indicated above, and wherein Y₁ and Y₂ further represent together a carbonyl group (═O), ═NH, ═N—OR₇, wherein R₇ is H, tosylate or branched or straight chain (C₁-C₆) alkyl, or Y₁ and Y₂ together is a group selected from: 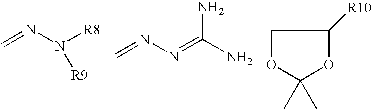
wherein R₈ and R₉ are the same or different and are H, branched or straight chain (C₁-C₆) alkyl, —(CH₂)₂—OH, CHO, CONH₂, tBOC (tert-Butoxycarbonyl), or mean —COCOOH, R₁₀ is H or CH3, and wherein when Y₁ is —O— (CH₂)₂—OH, Y₂ is OH, and wherein Z₁ is H, branched or straight chain (C₁-C₆) alkyl, (C₂-C₇) alkenyl, (C₂-C₇) alkynyl, tri-fluoroacetyl, formyl, phenyl or a group selected from: 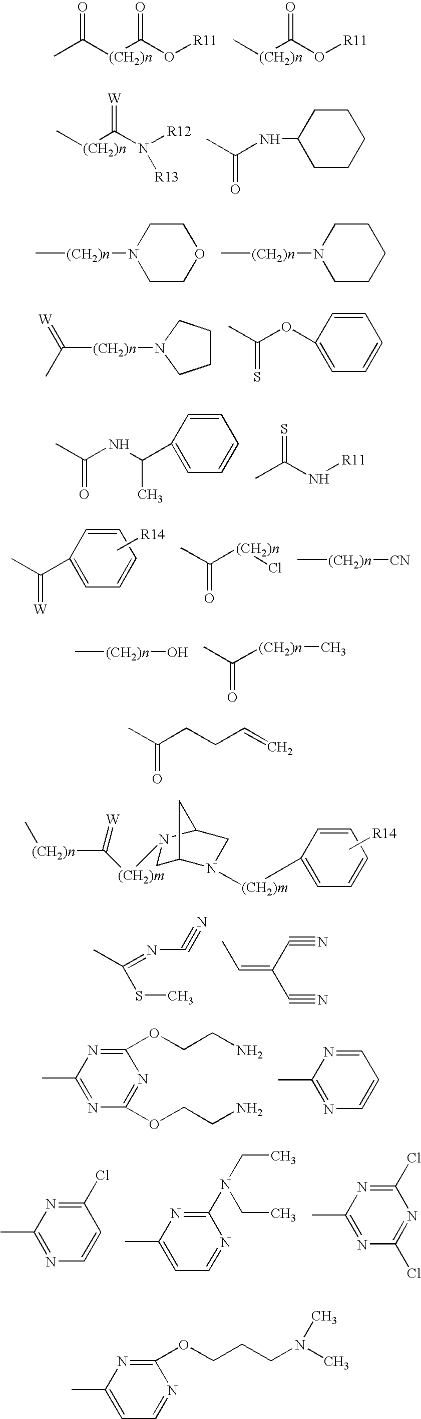 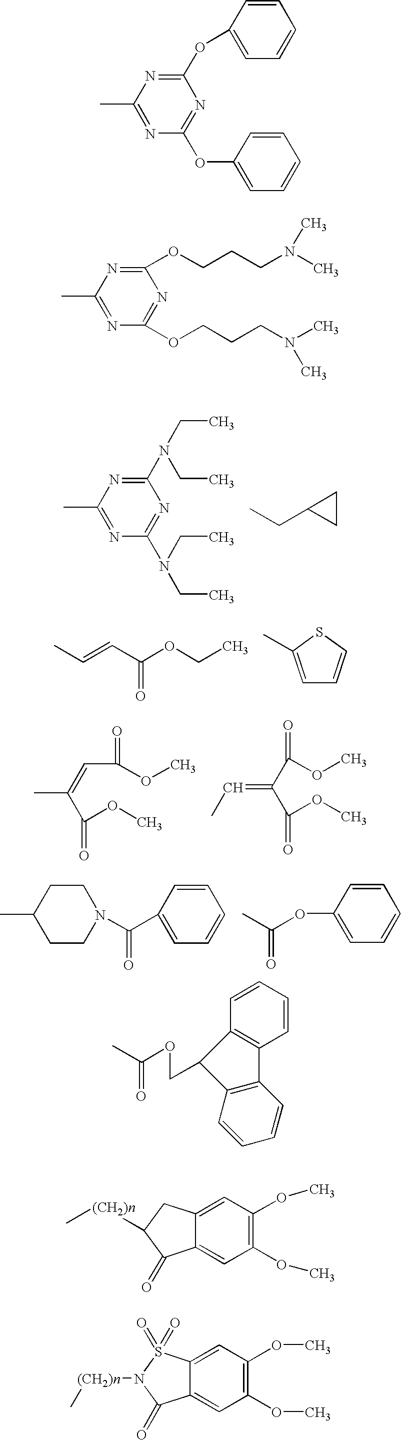 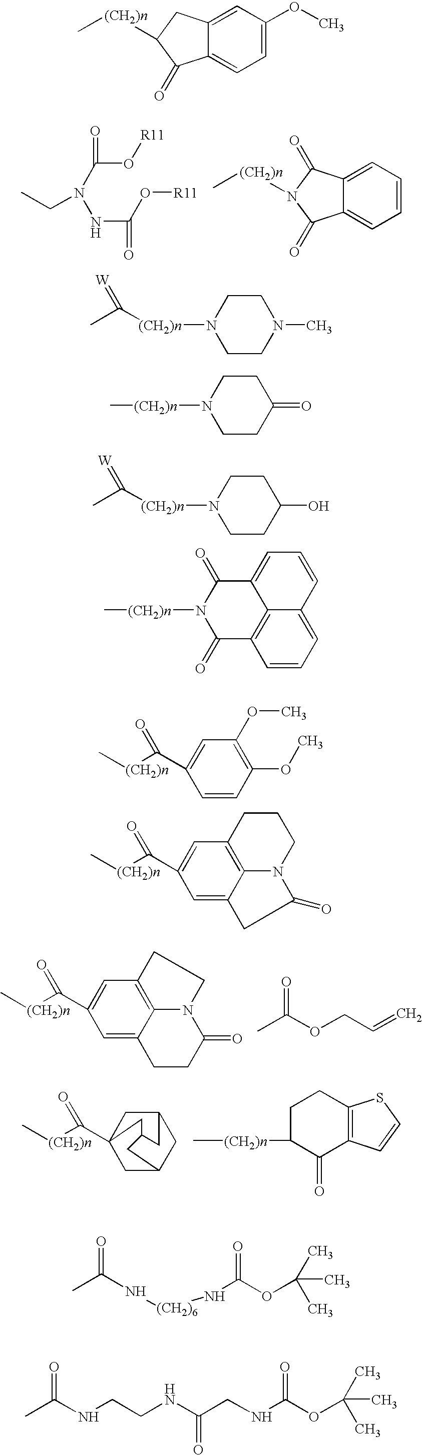 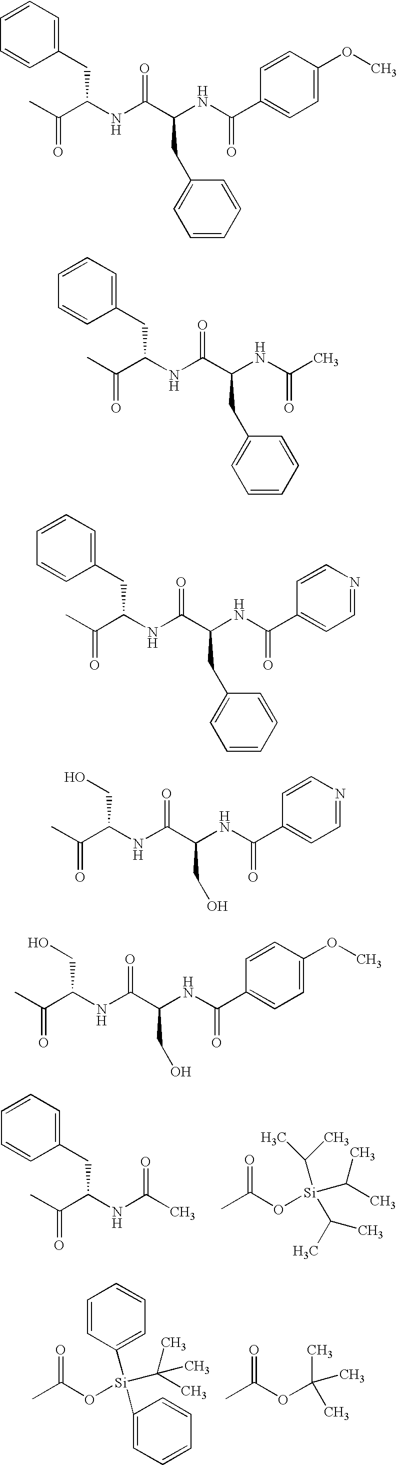
wherein R₁₁ is H, straight chain (C₁-C₆) alkyl, branched (C₁-C₆) alkyl or (C₂-C₇) alkenyl, R₁₂ and R₁₃ are the same or different and are selected from H, straight chain or branched (C₁-C₆) alkyl, phenyl, chlorophenyl, (trifluoromethyl)-phenyl or 1-naphtyl, wherein R₁₄ is H, F, CH₃, NO₂, Cl, Br, J, CF₃, n has the meaning indicated above, m is 0 or 1, and W has the meaning H or O, and wherein further Z₁ and R₃ form a common ring 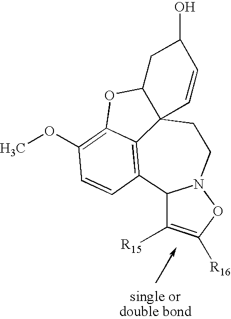
wherein R₁₅ and R₁₆ alternatively mean H, COOCH₃, COOCH₂CH₃, CN, COCH₃.

Other preferred galanthamine derivatives have the general formula Ib

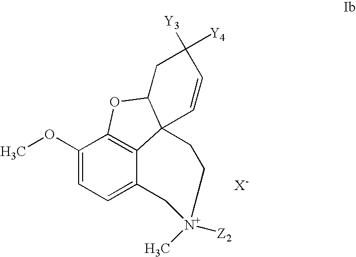

wherein Y₃ and Y₄ alternatively mean H and OH, X is Cl, Br or I, Z₂ is oxygen (N-oxide and no counterion), branched or straight chain (C₁-C₆) alkyl, or (C₂-C₇) alkenyl or (C₂-C₇) alkynyl or a group selected from: 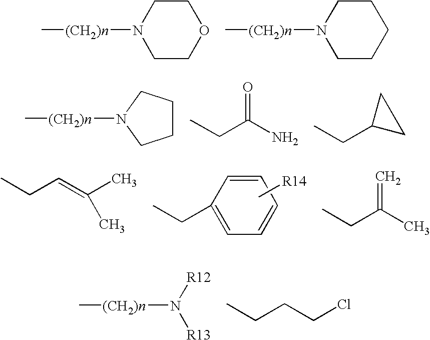
wherein n, R₁₂, R₁₃ and R₁₄ have the meanings as defined in accordance with claim 3.

Likewise other useful galanthamine derivatives of the invention that can be used have the general formula Ic 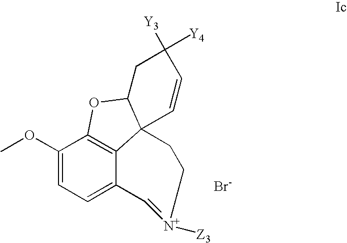
wherein Y₃ and Y₄ have the meanings defined above, and Z₃ is oxygen (N-oxide and no counterion) or is a methyl group.

Additional galanthamine derivatives used according to invention are further characterized by the general formula Id 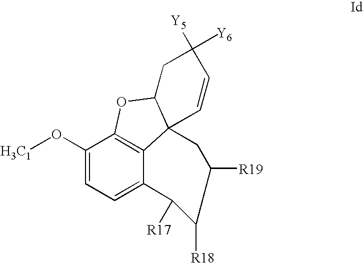
and their salts, wherein Y₅ and Y₆ alternatively are H or OH, or together form a keto group, and R₁₇, R₁₈, R₁₉ are alternatively for two substituents H, and wherein the third substituent is NH₂ or CONH₂.

Further preferable galanthamine derivatives that can be used according to the invention have the general formula Ie 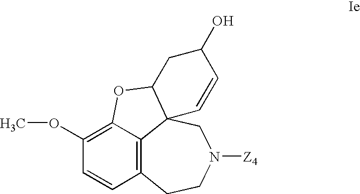
or their salts, wherein Z₄ is straight chain or branched (C₁-C₆) alkyl or 4-bromobenzyl.

Other preferable galanthamine derivatives that can be used according to the invention have the general formula If

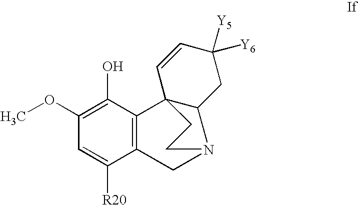

or their salts, wherein Y₅ and Y₆ have the meanings as defined above, and R₂₀ is H or Br.

The use of a galanthamine derivative with the following structural formula is particularly preferred 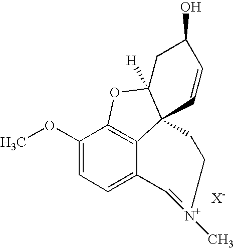
and its pharmaceutical acceptable salts, hydrates or solvates thereof and having the designated chemical name (4aS,6R,8aS)-6-Hydroxy-3-methoxy-11-methyl-4a,5,9,10-tetrahydro-6H-benzofuro[3a,3,2-f][2]benzazepinium.

The pharmaceutical acceptable salt counterions of (4aS,6R, 8aS)-6-Hydroxy-3-methoxy-11-methyl-4a,5,9,10-tetrahydro-6H-benzofuro[3a,3,2-ef][2]benzazepinium are selected from the group of halides, preferably bromide, carboxylic acids with 1-3 carboxyl functions, particularly preferably are tartrate, malonate, fumarate and succinate, and sulfonic acids, preferably methane sulfonic acid.

According to the invention the galanthamine as well as the galanthamine derivatives used in the present invention are prepared by known procedures in the art, like those described in WO 96/12692 A, WO 97/40049 and WO 01/74820. In the present invention the cholinergic activity of galanthamine and its derivatives is substantial, and this characteristic is going to be specified according to the invention using the inhibition of the cholinergic effect of cholinesterases. This characteristic can be shown on the following table—as the concentration values for acetyl and/or butyrylcholinesterase, lowered by 50% inhibition.

| | | | Acetyl- | Butyryl- |
|---|---|---|---|---|
| | | | cholinesterase | cholinesterase |
| Nr | STRUCTURE | stereo | IC-50 (μM) | IC-50 (μM) |
| 1 | 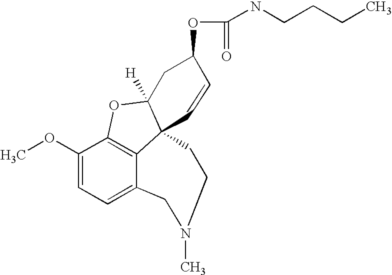 | (−) | >100 | 4.8 |
| 2 | 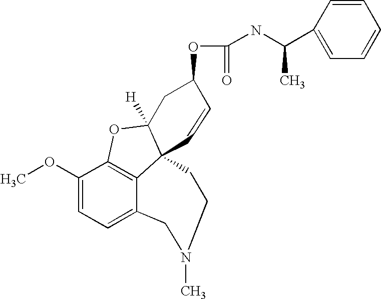 | (−) | | 70 |
| 3 | 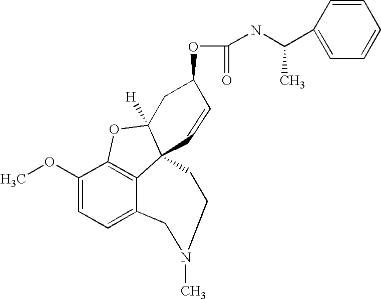 | (−) | | 75 |
| 4 | 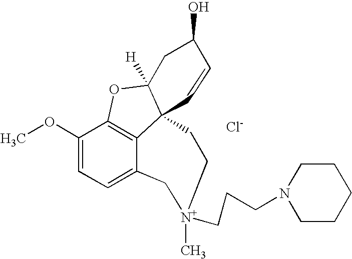 | (−) | 6 | |
| 5 | 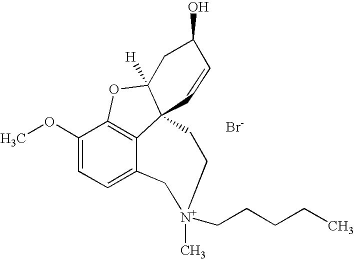 | (−) | | |
| 6 | 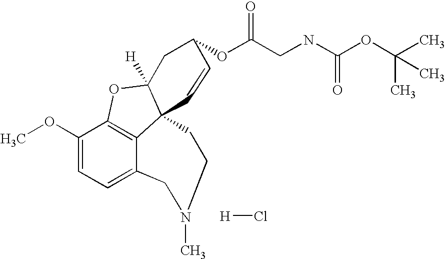 | (−) epi | 45 | |
| 7 | 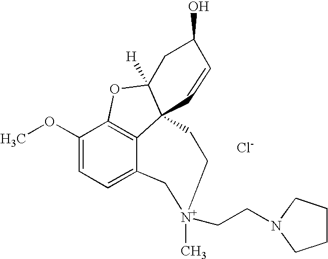 | (−) | 2 | |
| 8 | 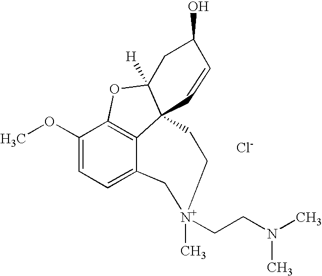 | (−) | 8 | |
| 9 | 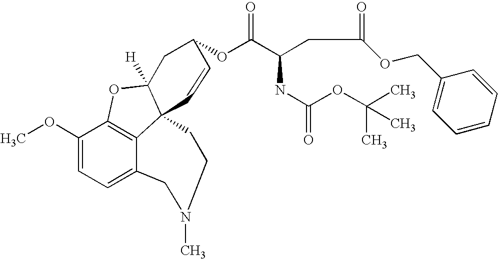 | (−) epi | | |
| 10 | 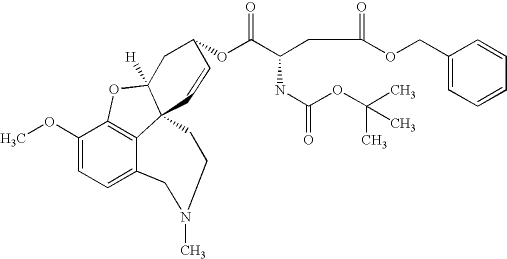 | (−) epi | | |
| 11 | 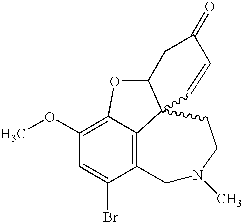 | (−/+) | 50 | |
| 12 | 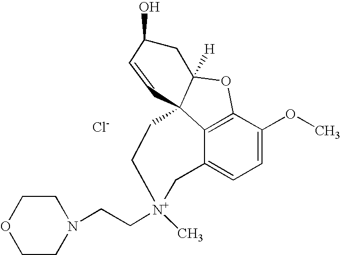 | (+) | 57 | 13 |
| 13 | 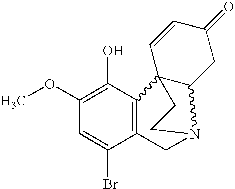 | (−/+) | 5 | |
| 14 | 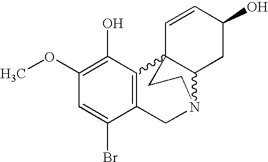 | (−/+) | >100 | 18 |
| 15 | 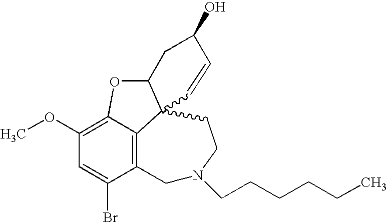 | (−/+) | 40 | 0.45 |
| 16 | 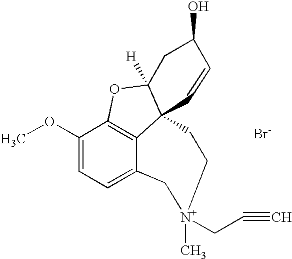 | (−) | 1.4 | 1.7 |
| 17 | 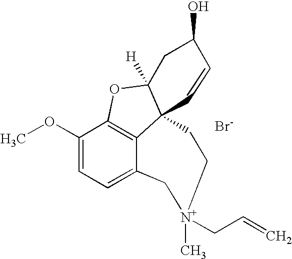 | (−) | | |
| 18 | 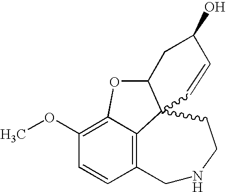 | (−/+) | 7 | |
| 19 | 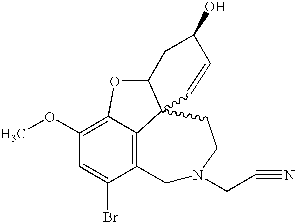 | (−/+) | >100 | 70 |
| 20 | 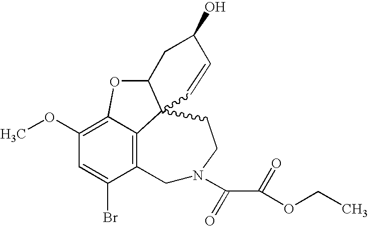 | (−/+) | 32 | 11 |
| 21 | 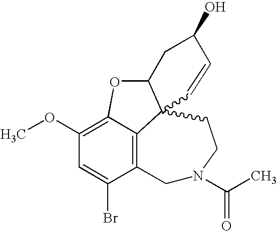 | (−/+) | | |
| 22 | 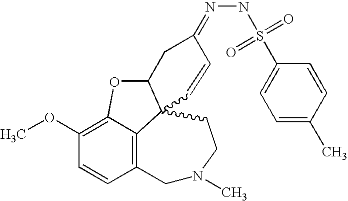 | (−/+) | | |
| 23 | 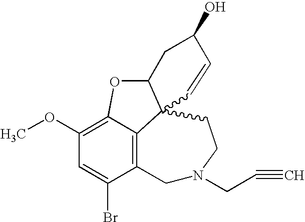 | (−/+) | 63 | 10 |
| 24 | 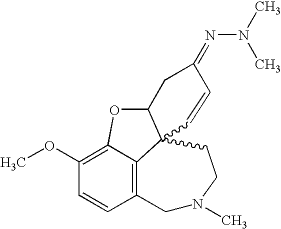 | (−/+) | 80 | 60 |
| 25 | 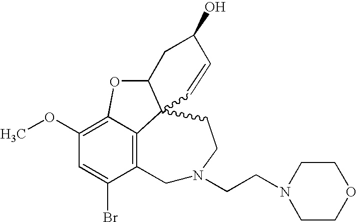 | (−/+) | 3 | |
| 26 | 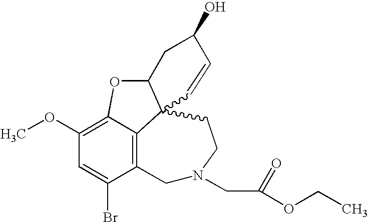 | (−/+) | 20 | |
| 27 | 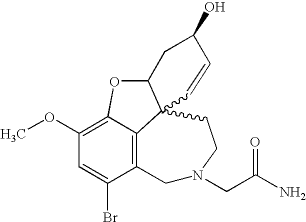 | (−/+) | >100 | 15 |
| 28 | 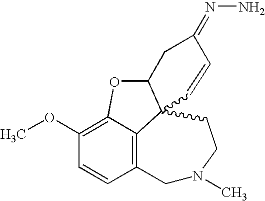 | (−/+) | 40 | |
| 29 | 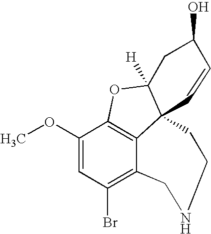 | (−) | 3 | |
| 30 | 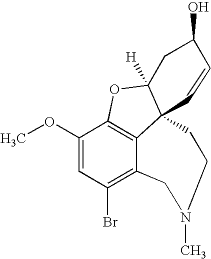 | (−) | 4 | |
| 31 | 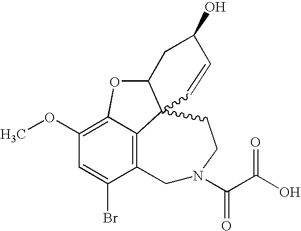 | (−/+) | | |
| 32 | 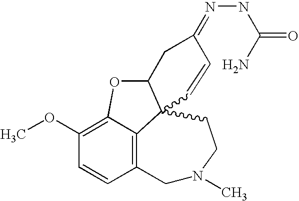 | (−/+) | >100 | 20 |
| 33 | 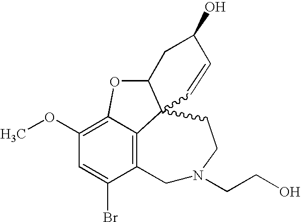 | (−/+) | 34 | 6.4 |
| 34 | 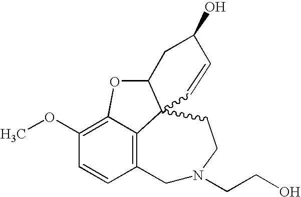 | (−/+) | 14 | 26 |
| 35 | 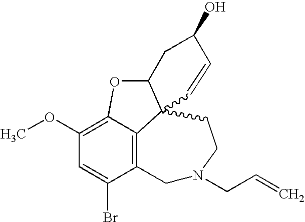 | (−/+) | >100 | 2.6 |
| 36 | 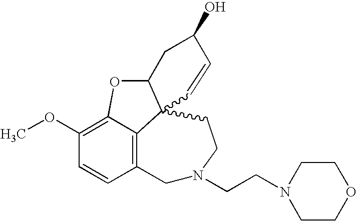 | (−/+) | 13 | 7 |
| 37 | 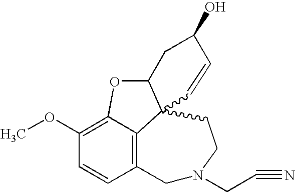 | (−/+) | 30 | >100 |
| 38 | 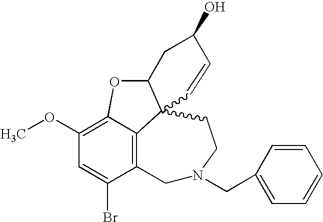 | (−/+) | >100 | 0.24 |
| 39 | 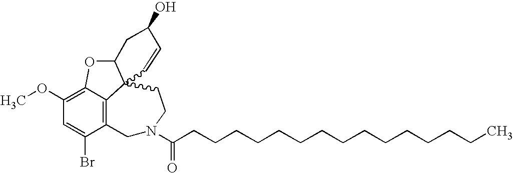 | (−/+) | | |
| 40 | 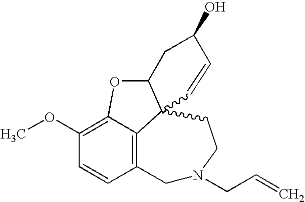 | (−/+) | 3.3 | 3.1 |
| 41 | 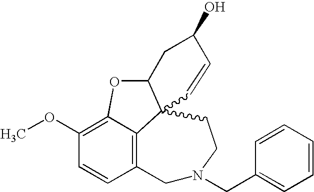 | (−/+) | 0.7 | 0.65 |
| 42 | 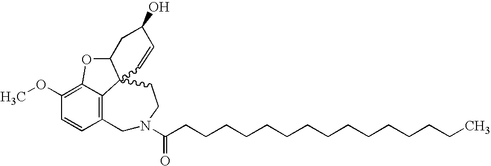 | (−/+) | | |
| 43 | 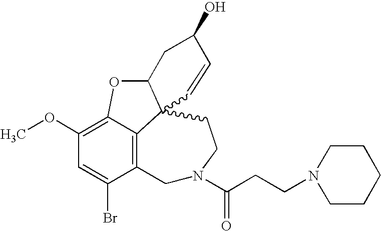 | (−/+) | 0.2 | |
| 44 | 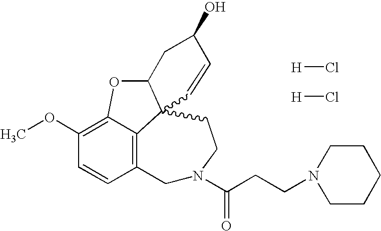 | (−/+) | | |
| 45 | 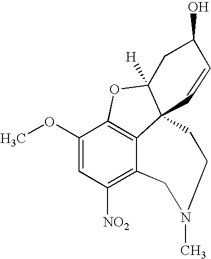 | (−) | >100 | 25 |
| 46 | 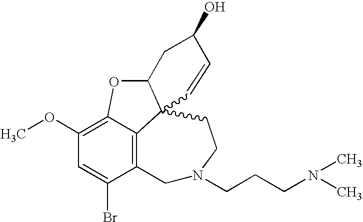 | (−/+) | | |
| 47 | 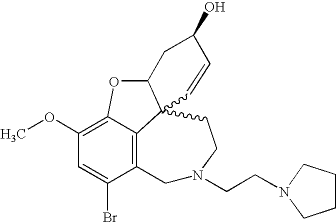 | (−/+) | | |
| 48 | 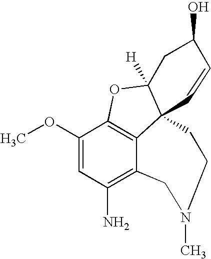 | (−) | 77 | 4.9 |
| 49 | 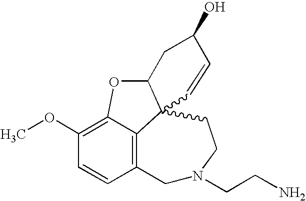 | (−/+) | | |
| 50 | 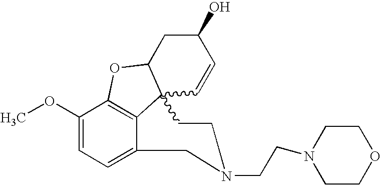 | (+/−) | | |
| 51 | 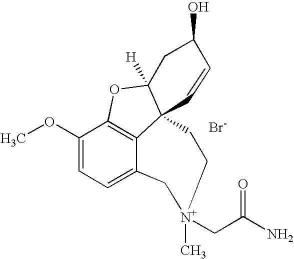 | (−) | 3.1 | 2.5 |
| 52 | 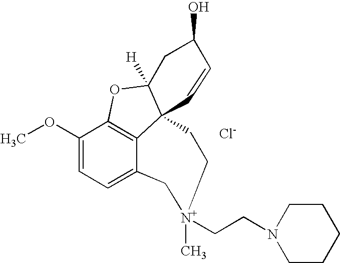 | (−) | 4 | |
| 53 | 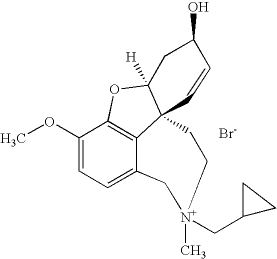 | (−) | 1.2 | 3.6 |
| 54 | 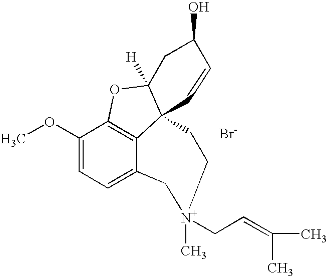 | (−) | 0.2 | 0.21 |
| 55 | 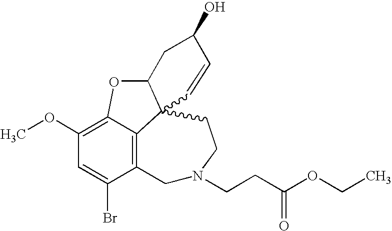 | (−/+) | >100 | 19 |
| 56 | 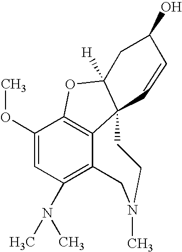 | (−) | >100 | 0.47 |
| 57 | 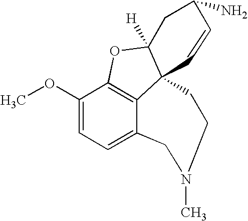 | (−) epi | | |
| 58 | 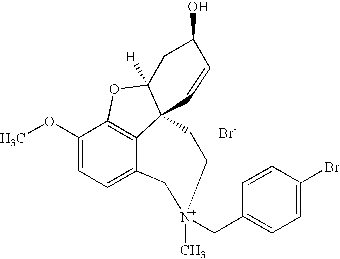 | (−) | 0.2 | 0.6 |
| 59 | 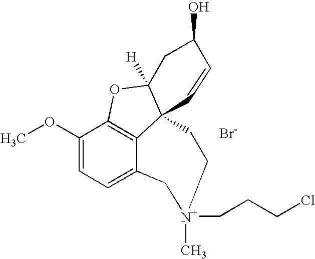 | (−) | 0.35 | 4.4 |
| 60 | 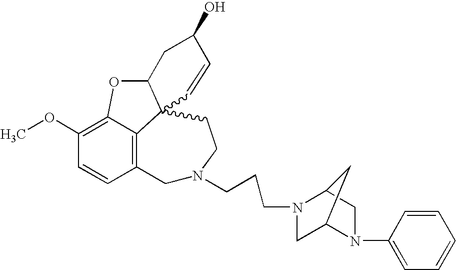 | (−/+) | 24 | 7.5 |
| 61 | 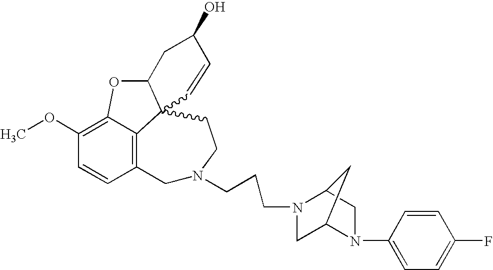 | (−/+) | 5.2 | 5 |
| 62 | 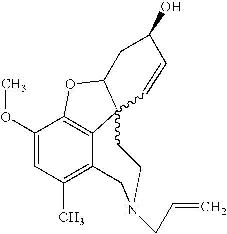 | (−/+) | >100 | 2.3 |
| 63 | 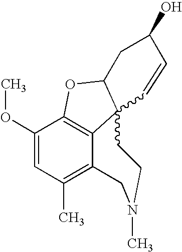 | (−/+) | >100 | 17 |
| 64 | 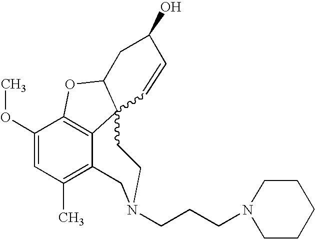 | (−/+) | 46 | 0.6 |
| 65 | 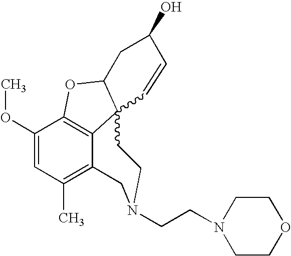 | (−/+) | >100 | 5.2 |
| 66 | 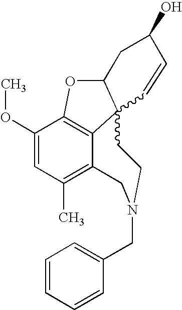 | (−/+) | | |
| 67 | 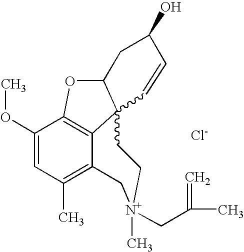 | (−/+) | 70 | 2.4 |
| 68 | 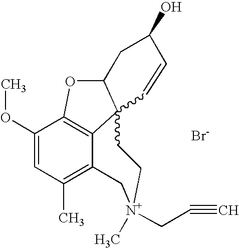 | (−/+) | 78 | 2.5 |
| 69 | 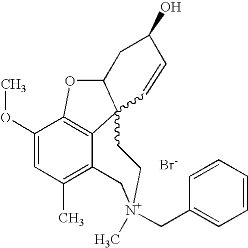 | (−/+) | 47 | 0.7 |
| 70 | 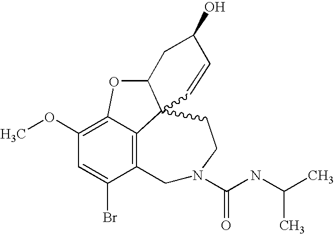 | (−/+) | >100 | 25 |
| 71 | 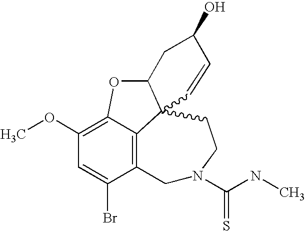 | (−/+) | 31 | 20 |
| 72 | 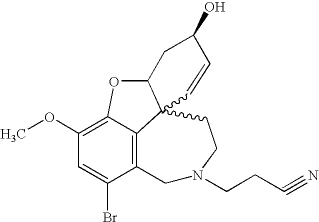 | (−/+) | >100 | 43 |
| 73 | 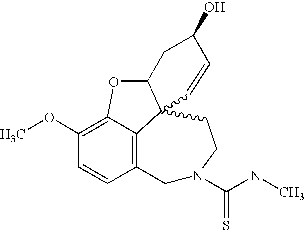 | (−/+) | 23 | 30 |
| 74 | 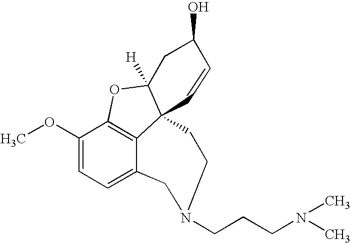 | (−/+) | 6 | 10 |
| 75 | 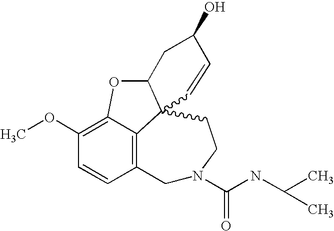 | (−/+) | 4.2 | >100 |
| 76 | 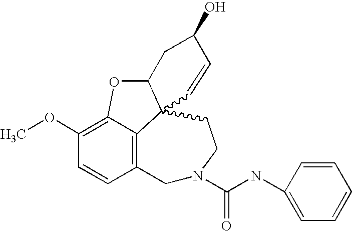 | (−/+) | 70 | >100 |
| 77 | 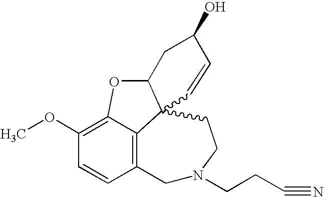 | (−/+) | 90 | >100 |
| 78 | 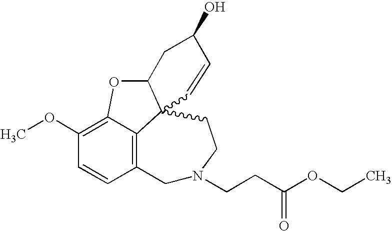 | (−/+) | 9.5 | 17 |
| 79 | 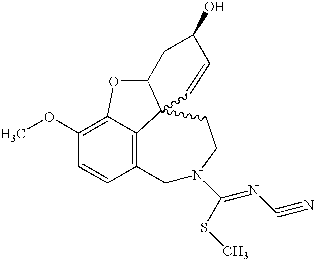 | (−/+) | 25 | 0.54 |
| 80 | 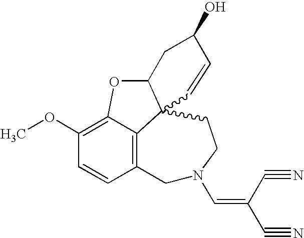 | (−/+) | 28.5 | >100 |
| 81 | 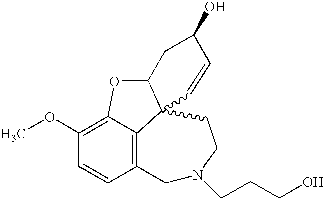 | (−/+) | 7.2 | 21 |
| 82 | 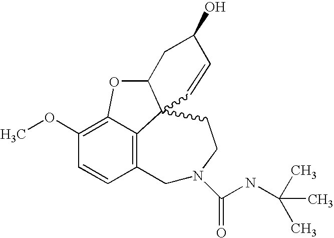 | (−/+) | 4.8 | >100 |
| 83 | 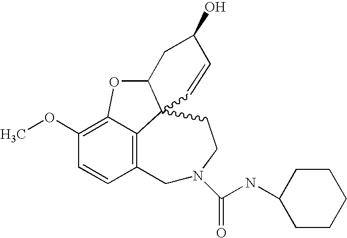 | (−/+) | 6.7 | >100 |
| 84 | 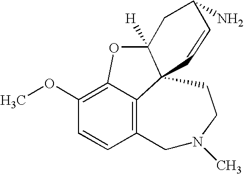 | (−) epi | 40 | 6 |
| 85 | 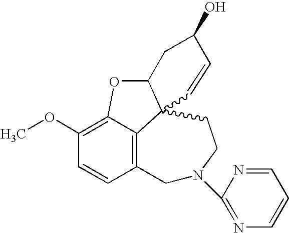 | (−/+) | 38 | 30 |
| 86 | 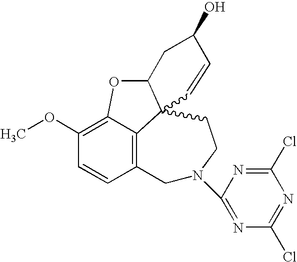 | (−/+) | | |
| 87 | 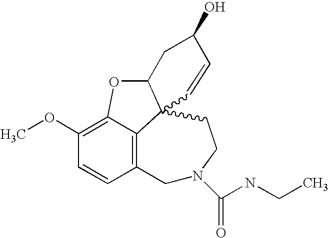 | (−/+) | 33 | >100 |
| 88 | 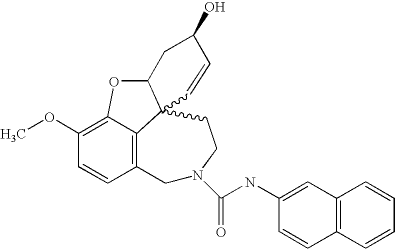 | (−/+) | 36 | >100 |
| 89 | 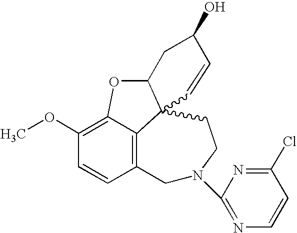 | (−/+) | 66 | >100 |
| 90 | 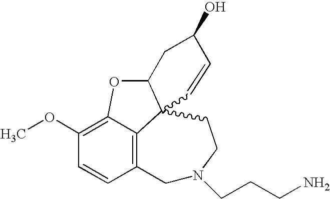 | (−/+) | 3.4 | 11 |
| 91 | 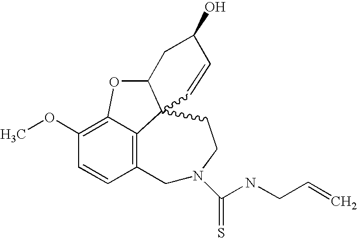 | (−/+) | 21 | >100 |
| 92 | 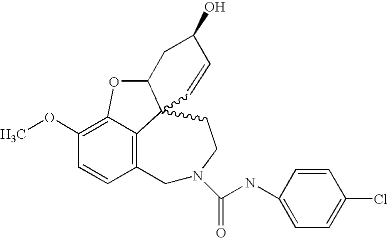 | (−/+) | 24 | >100 |
| 93 | 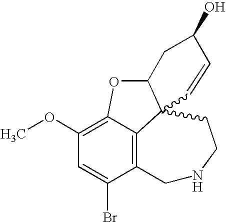 | (−/+) | 5 | |
| 94 | 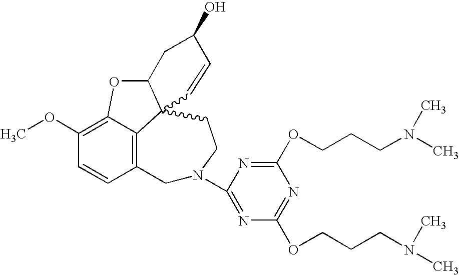 | (−/+) | 70 | 40 |
| 95 | 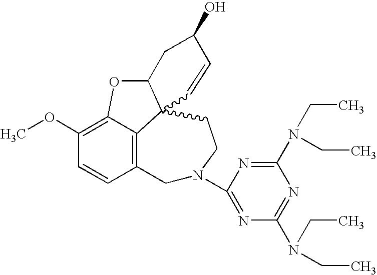 | (−/+) | 40 | >100 |
| 96 | 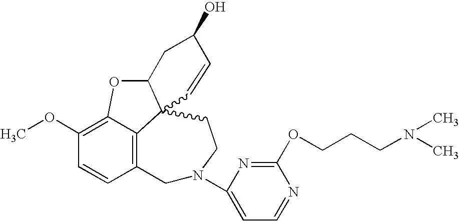 | (−/+) | 7.4 | 36 |
| 97 | 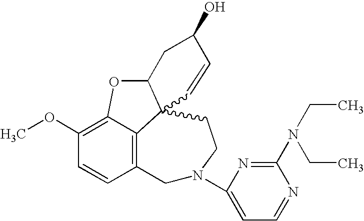 | (−/+) | 25 | >100 |
| 98 | 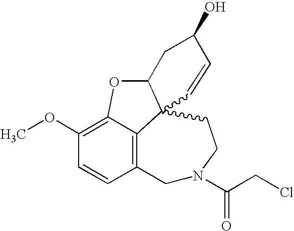 | (−/+) | 17.5 | 20 |
| 99 | 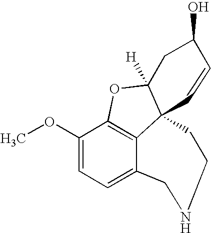 | (−) | 2.4 | 4 |
| 100 | 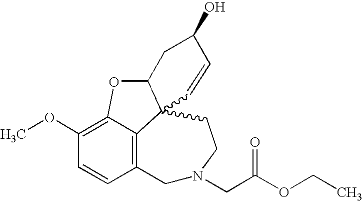 | (−/+) | 40 | 90 |
| 101 | 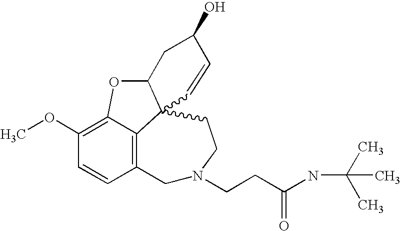 | (−/+) | 45 | 26 |
| 102 | 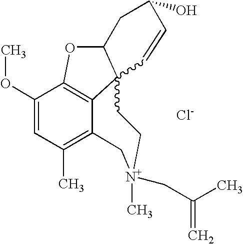 | (−/+) epi | >100 | 95 |
| 103 | 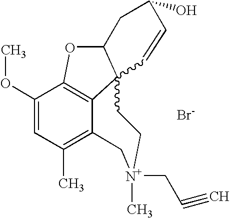 | (−/+) epi | 59 | 45 |
| 104 | 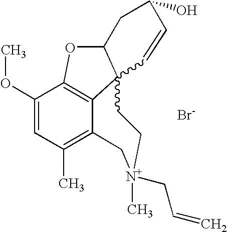 | (−/+) epi | >100 | 52 |
| 105 | 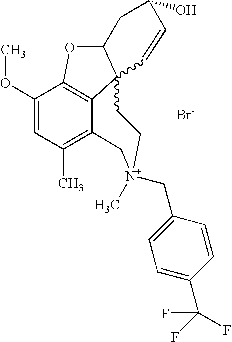 | (−/+) epi | 60 | 5.4 |
| 106 | 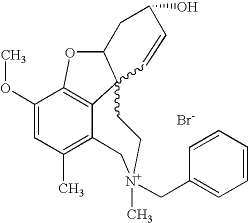 | (−/+) epi | >100 | 3 |
| 107 | 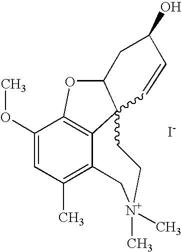 | (−/+) | >100 | 14 |
| 108 | 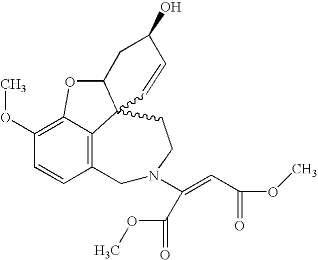 | (−/+) | 140 | 80 |
| 109 | 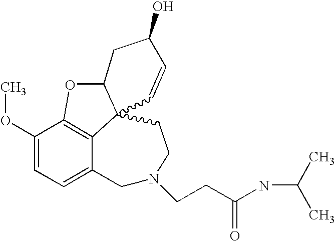 | (−/+) | 54.5 | 36 |
| 110 | 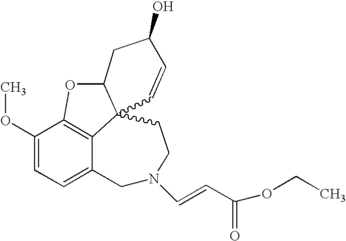 | (−/+) | 50 | >100 |
| 111 | 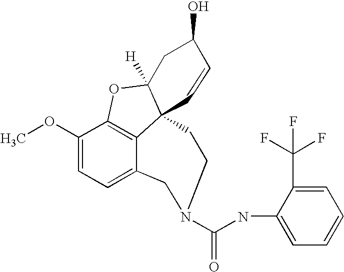 | (−) | 30 | >100 |
| 112 | 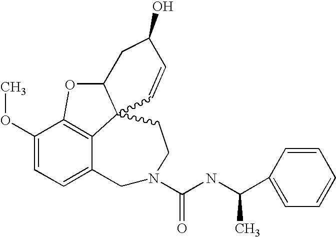 | (−/+) | 30 | >100 |
| 113 | 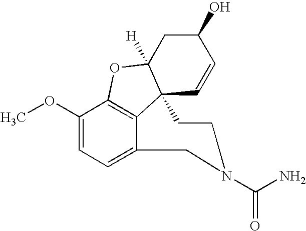 | (−) | 44 | >100 |
| 114 | 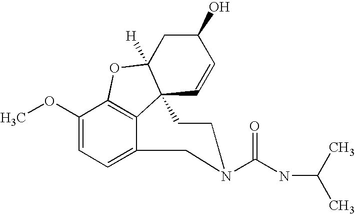 | (−) | 2.6 | 10 |
| 115 | 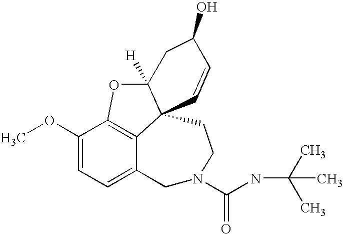 | (−) | 2.5 | 7 |
| 116 | 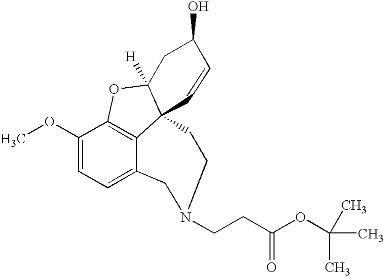 | (−) | 15 | 4 |
| 117 | 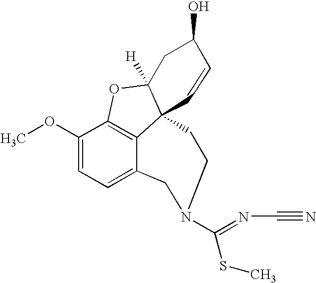 | (−) | 6.7 | 30 |
| 118 | 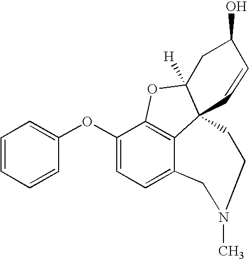 | (−) | 21 | 3.4 |
| 119 | 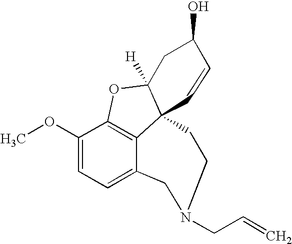 | (−) | | |
| 120 | 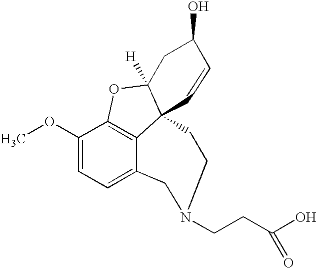 | (−) | 42 | 40 |
| 121 | 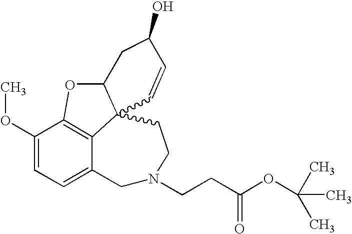 | (−/+) | 33 | 7.3 |
| 122 | 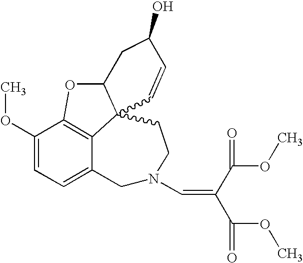 | (−/+) | 100 | 32 |
| 123 | 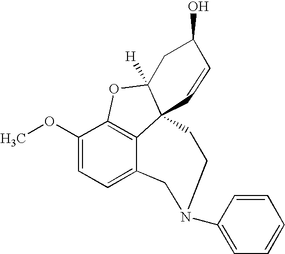 | (−) | 0.5 | 0.24 |
| 124 | 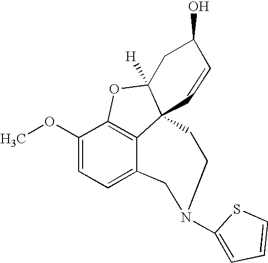 | (−) | 4 | 0.54 |
| 125 | 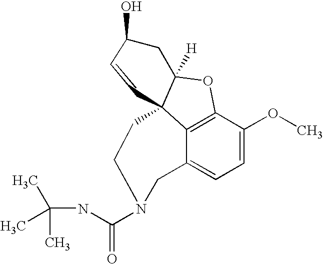 | (+) | 93 | 100 |
| 126 | 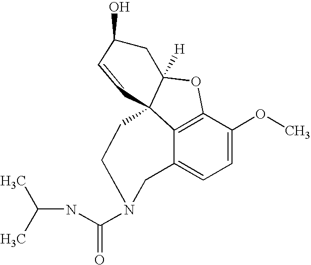 | (+) | 8 | 90 |
| 127 | 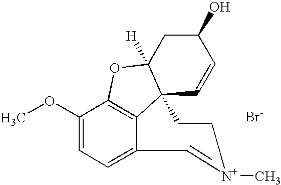 | (−) | 0.3 | 1.5 |
| 128 | 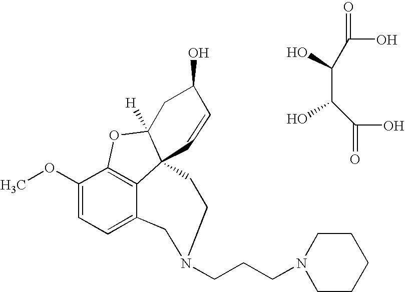 | (−) | 0.3 | 1.5 |
| 129 | 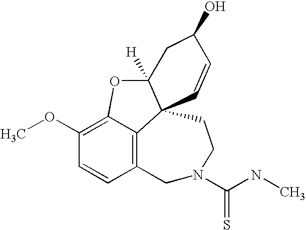 | (−) | 18.5 | 63 |
| 130 | 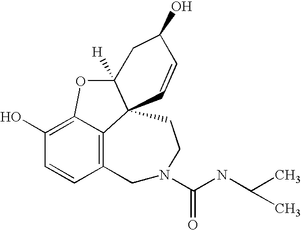 | (−) | 6.3 | 60 |
| 131 | 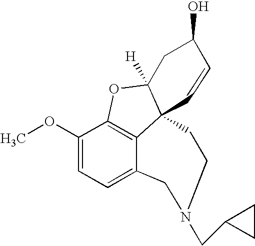 | (−) | 0.7 | 1.2 |
| 132 | 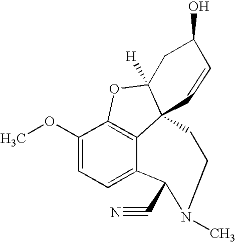 | (−) | 1.2 | 100 |
| 133 | 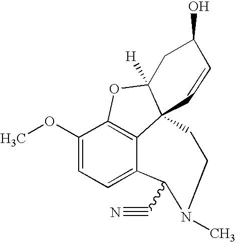 | (−) | 0.8 | >100 |
| 134 | 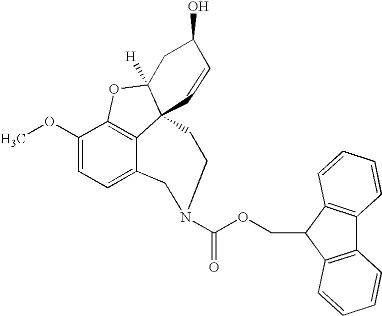 | (−) | 40 | 100 |
| 135 | 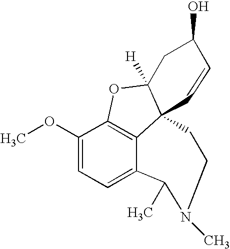 | (−) | 4.2 | 25 |
| 136 | 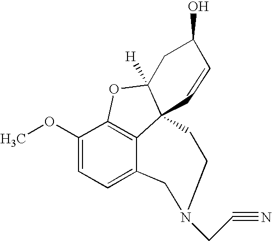 | (−) | 15 | 32 |
| 137 | 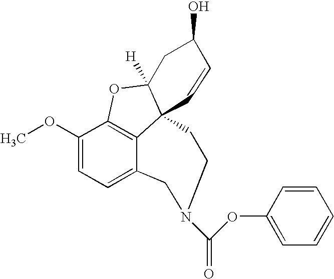 | (−) | 46 | >100 |
| 138 | 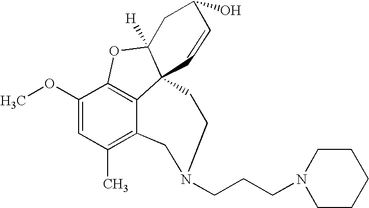 | (−) epi | >100 | 70 |
| 139 | 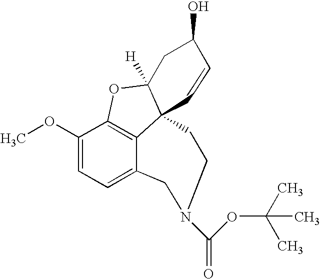 | (−) | 23 | >100 |
| 140 | 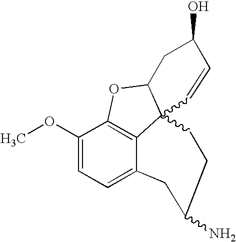 | (+/−) | 5.3 | >100 |
| 141 | 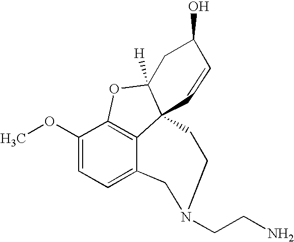 | (−) | 1.3 | 2.1 |
| 142 | 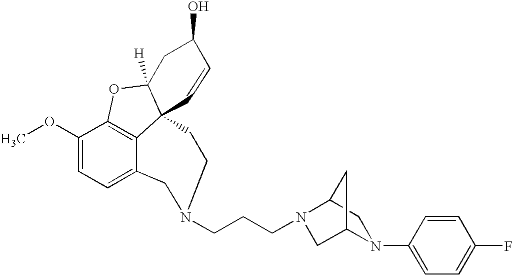 | (−) | 3 | 2.4 |
| 143 | 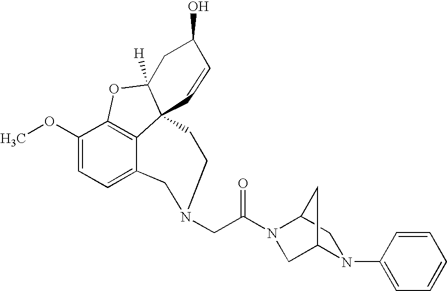 | (−) | 8.4 | 2.4 |
| 144 | 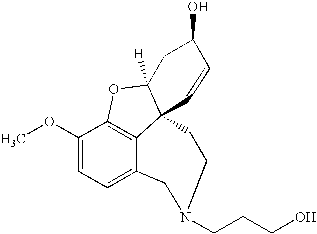 | (−) | 2.8 | 5 |
| 145 | 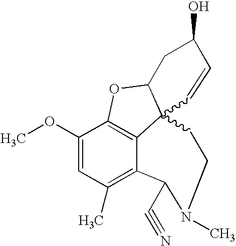 | (+/−) | 80 | >100 |
| 146 | 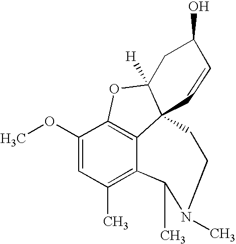 | (−) | 83 | 30 |
| 147 | 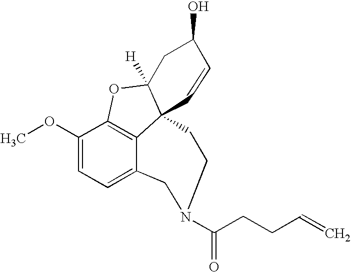 | (−) | 8.4 | 2.6 |
| 148 | 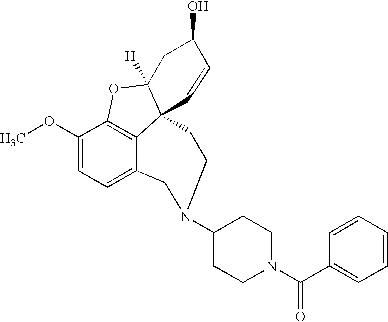 | (−) | 24 | 3 |
| 149 | 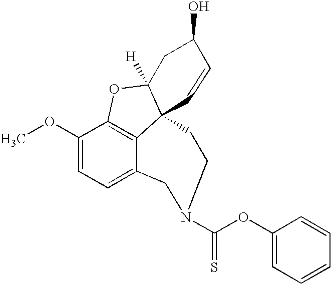 | (−) | 7.2 | >100 |
| 150 | 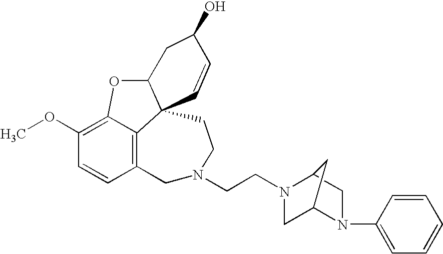 | (−) | 2.9 | 0.85 |
| 151 | 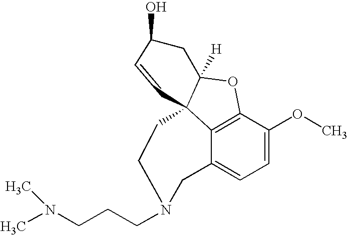 | (+) | 64 | 67 |
| 152 | 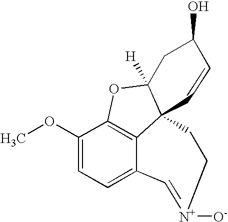 | (−) | 50 | >100 |
| 153 | 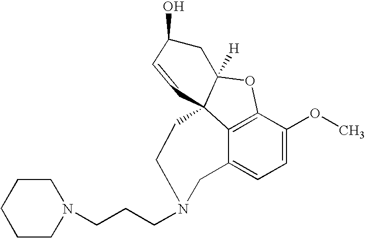 | (+) | 9 | 23 |
| 154 | 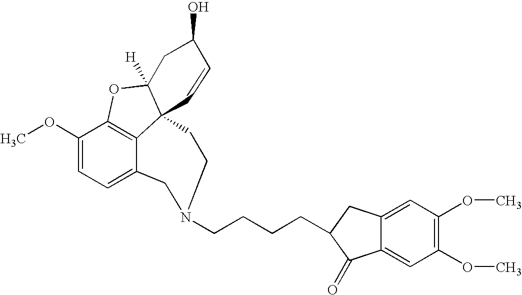 | (−) | 0.02 | 0.8 |
| 155 | 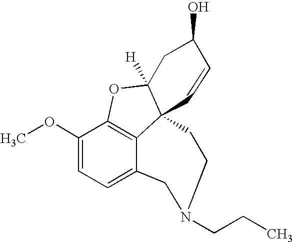 | (−) | 0.3 | 1.5 |
| 156 | 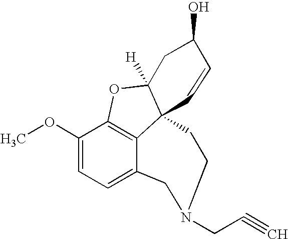 | (−) | 32 | 30 |
| 157 | 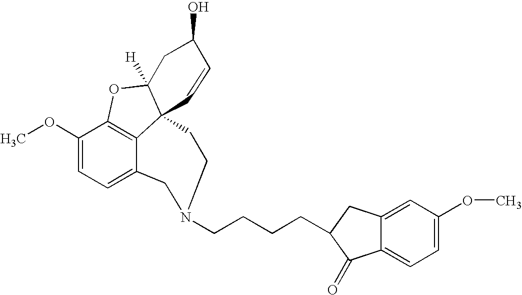 | (−) | 0.022 | 0.8 |
| 158 | 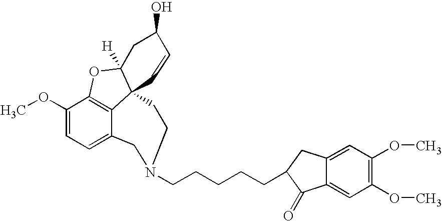 | (−) | 0.0052 | 0.24 |
| 159 | 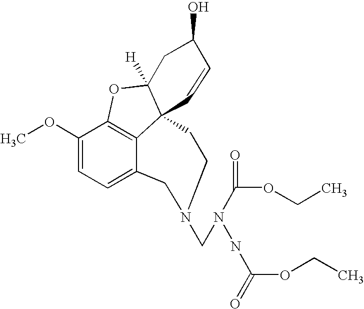 | (−) | 3 | >100 |
| 160 | 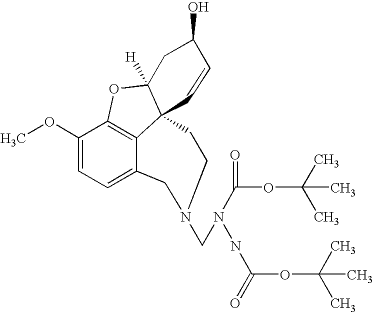 | (−) | 3.6 | 20 |
| 161 | 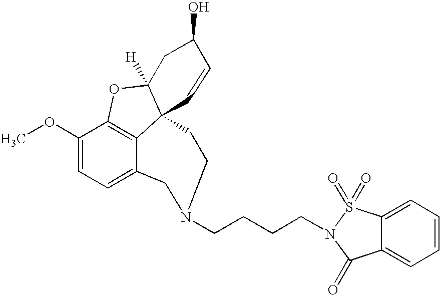 | (−) | 0.022 | 1.5 |
| 162 | 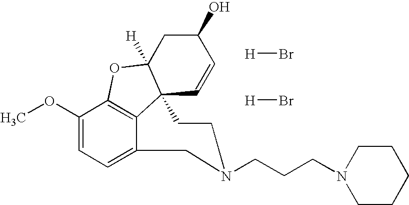 | (−) | 0.36 | |
| 163 | 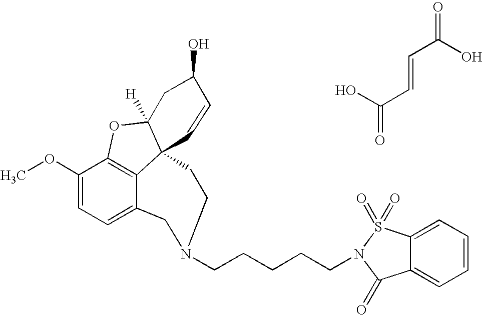 | (−) | 0.022 | |
| 164 | 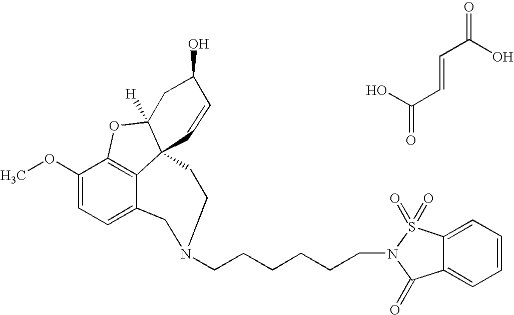 | (−) | 0.043 | |
| 165 | 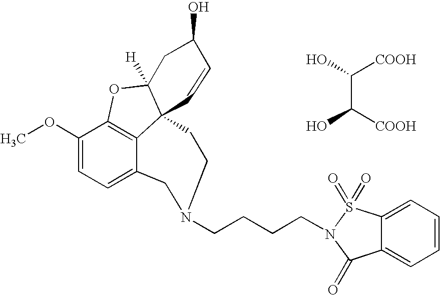 | (−) | 0.027 | |
| 166 | 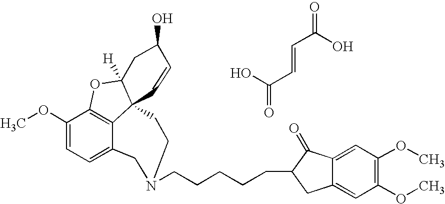 | (−) | 0.023 | |
| 167 | 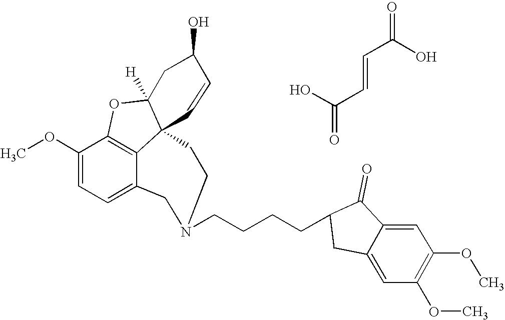 | (−) | 0.02 | |
| 168 | 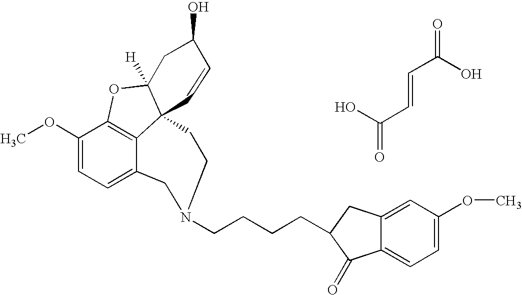 | (−) | 0.024 | |
| 169 | 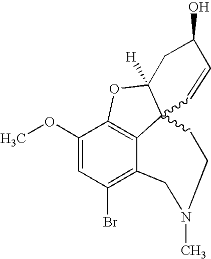 | (+/−) | | |
| 170 | 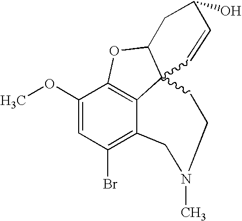 | (+/−) epi | | |
| 171 | 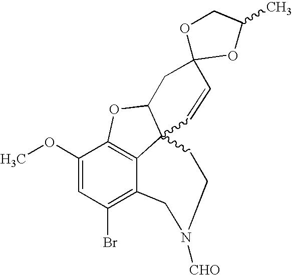 | (+/−) | | |
| 172 | 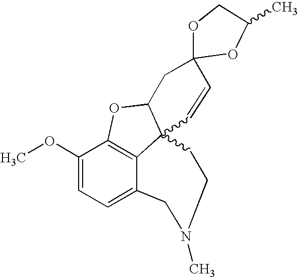 | (+/−) | | |
| 173 | 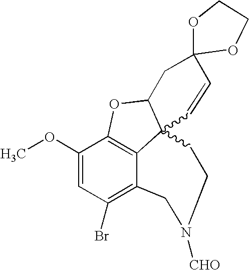 | | | |
| 174 | 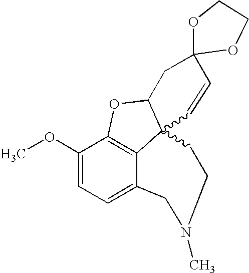 | (+/−) | | |
| 175 | 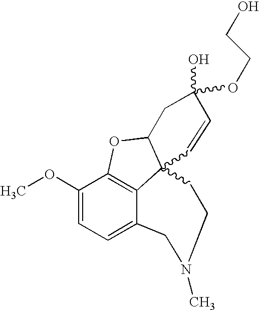 | (+/−) | | |
| 176 | 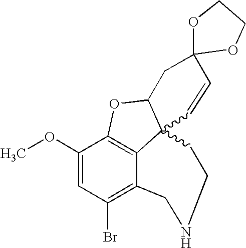 | (+/−) | | |
| 177 | 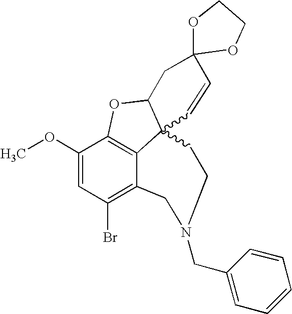 | (+/−) | | |
| 178 | 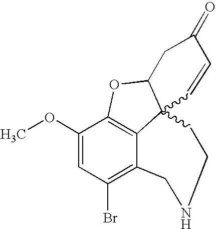 | (+/−) | | |
| 179 | 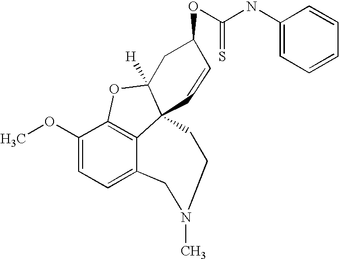 | (−) | 51 | 30 |
| 180 | 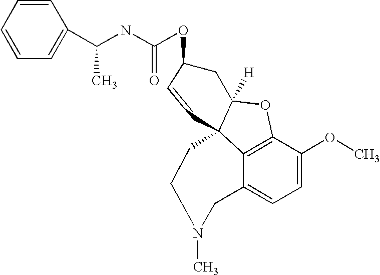 | (+) | 85 | |
| 181 | 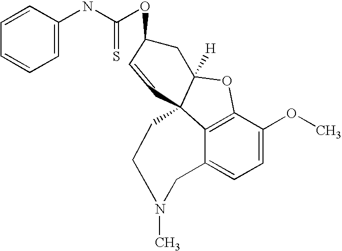 | (+) | 35 | |
| 182 | 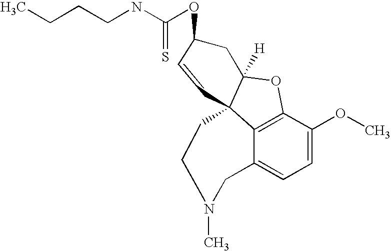 | (+) | 85 | |
| 183 | 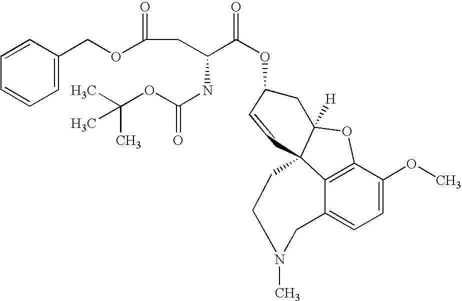 | (+) epi | | |
| 184 | 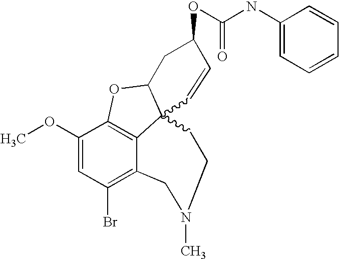 | (+/−) | | |
| 185 | 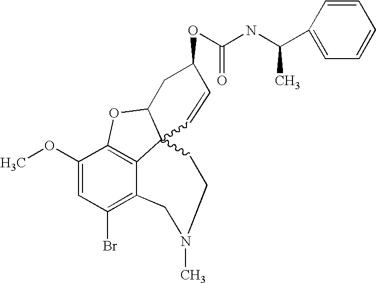 | (+/−) | | |
| 186 | 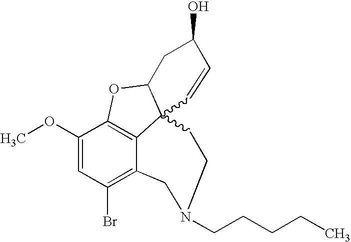 | (+/−) | | |
| 187 | 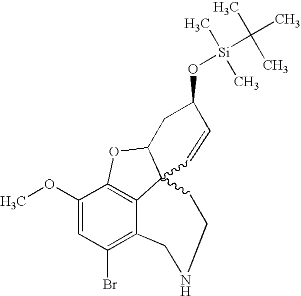 | (+/−) | | |
| 188 | 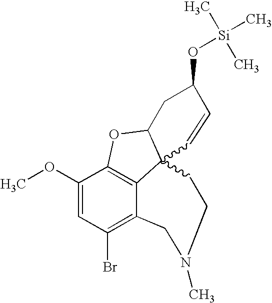 | (+/−) | | |
| 189 | 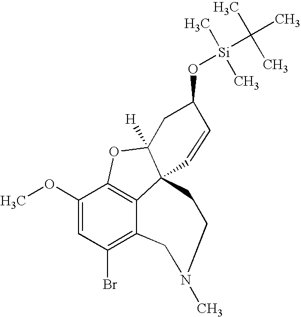 | (−) | | |
| 190 | 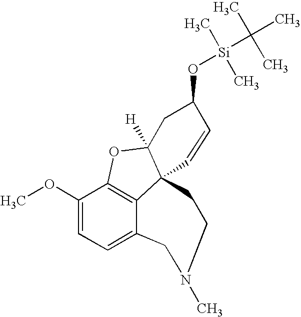 | (−) | | |
| 191 | 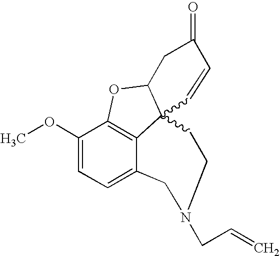 | (+/−) | | |
| 192 | 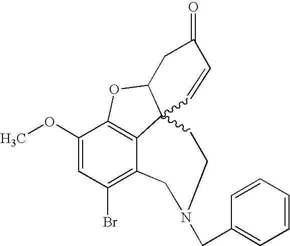 | (+/−) | | |
| 193 | 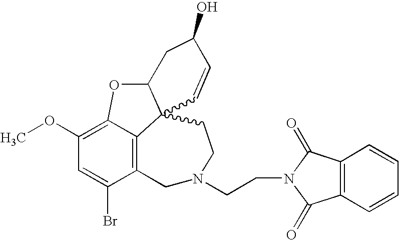 | (+/−) | | |
| 194 | 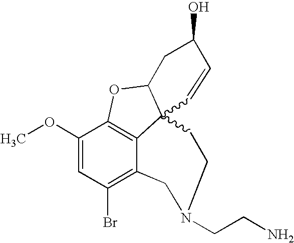 | (+/−) | | |
| 195 | 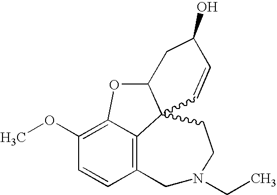 | (+/−) | | |
| 196 | 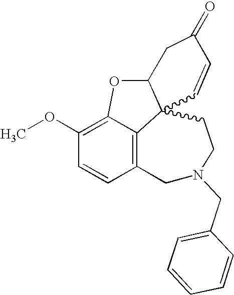 | (+/−) | | |
| 197 | 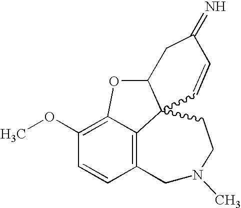 | (+/−) | | |
| 198 | 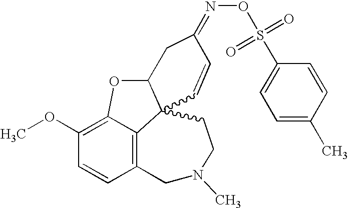 | (+/−) | | |
| 199 | 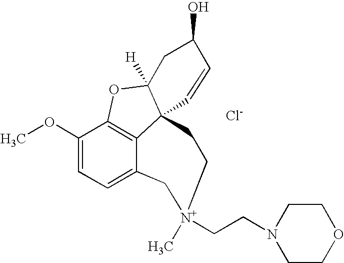 | (−) | 5 | |
| 200 | 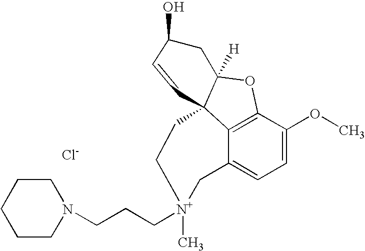 | (+) | | |
| 201 | 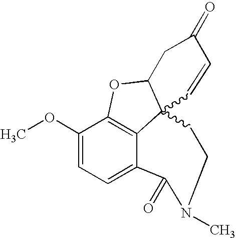 | (+/−) | | |
| 202 | 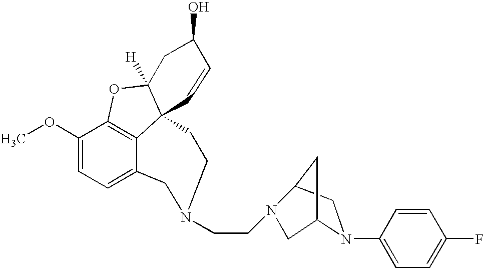 | (−) | | |
| 203 | 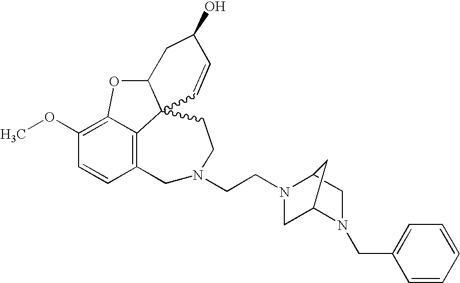 | (+/−) | | |
| 204 | 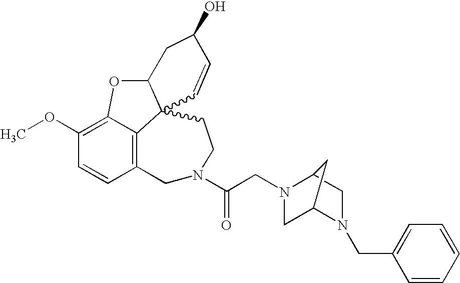 | (+/−) | | |
| 205 | 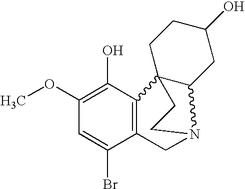 | (+/−) | 50 | |
| 206 | 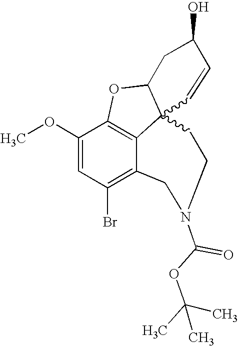 | (+/−) | | |
| 207 | 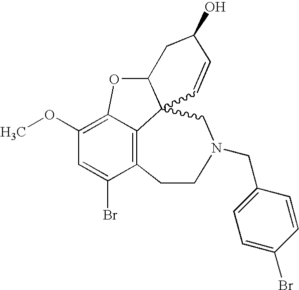 | (+/−) | | |
| 208 | 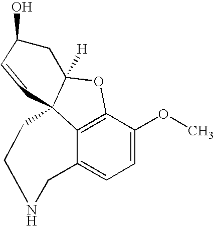 | (+) | | |
| 209 | 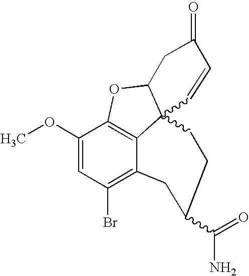 | (+/−) | | |
| 210 | 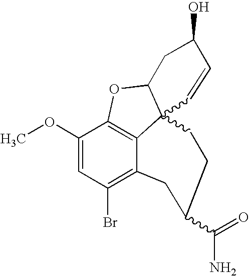 | (+/−) | | |
| 211 | 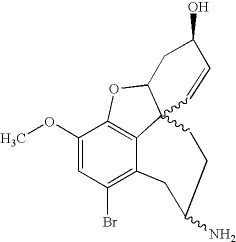 | (+/−) | | |
| 212 | 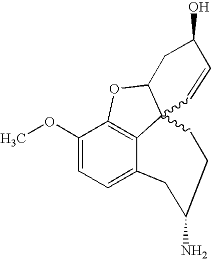 | (+/−) | | |
| 213 | 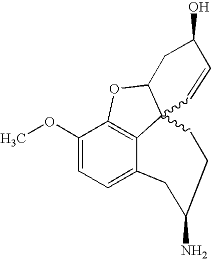 | (+/−) | | |
| 214 | 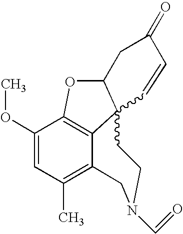 | (+/−) | | |
| 215 | 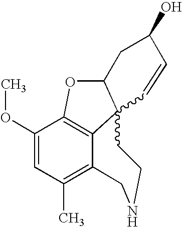 | (+/−) | | |
| 216 | 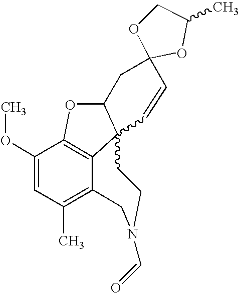 | (+/−) | | |
| 217 | 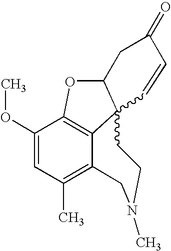 | (+/−) | | |
| 218 | 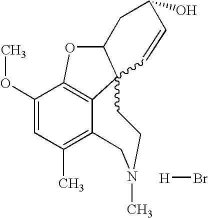 | (+/−) epi | | |
| 219 | 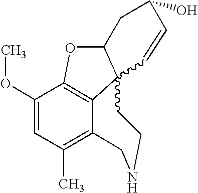 | (+/−) epi | | |
| 220 | 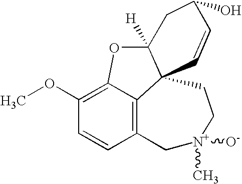 | (−) epi | | |
| 221 | 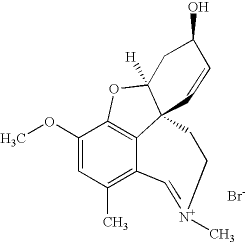 | (−) | | |
| 222 | 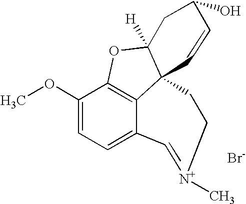 | (−) epi | | |
| 223 | 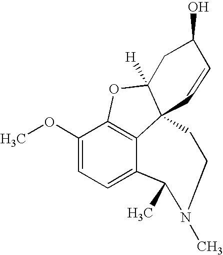 | (−) | | |
| 224 | 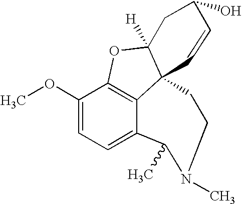 | epi | | |
| 225 | 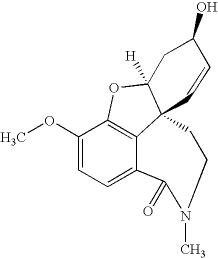 | (−) | | |
| 226 | 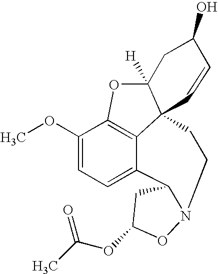 | (−) | | |
| 227 | 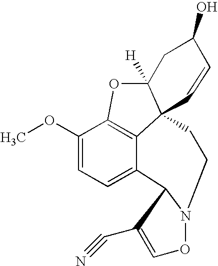 | (−) | | |
| 228 | 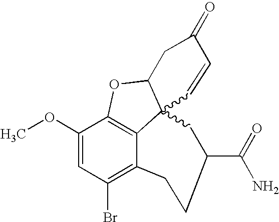 | (+/−) | | |
| 229 | 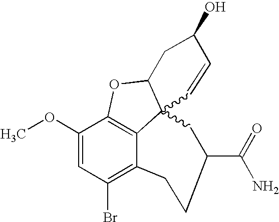 | (+/−) | | |
| 230 | 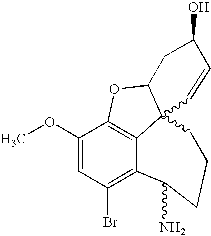 | (+/−) | | |
| 231 | 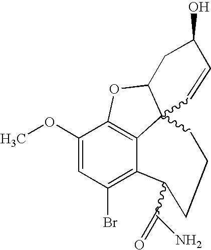 | (+/−) | | |
| 232 | 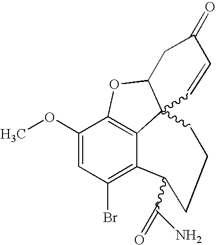 | (+/−) | | |
| 233 | 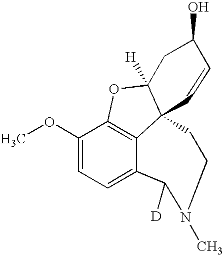 | (−) | | |
| 234 | 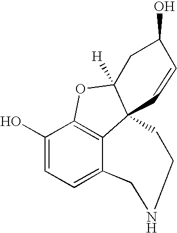 | (−) | | |
| 235 | 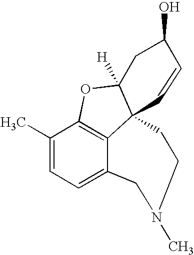 | (−) | | |
| 236 | 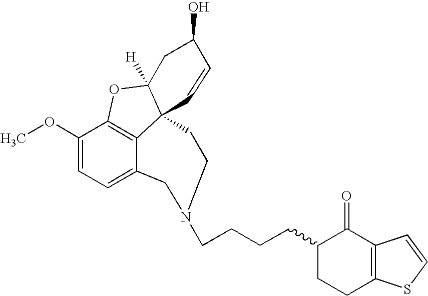 | (−) | | |
| 237 | 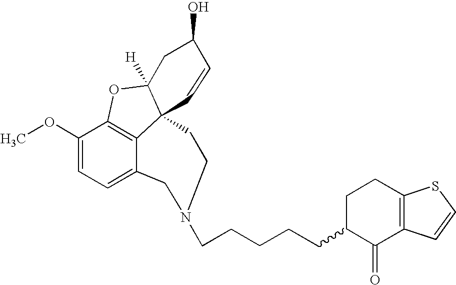 | (−) | | |
| 238 | 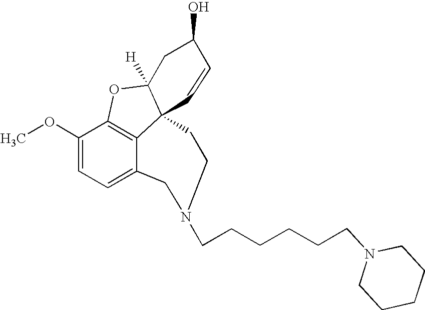 | (−) | | |
| 239 | 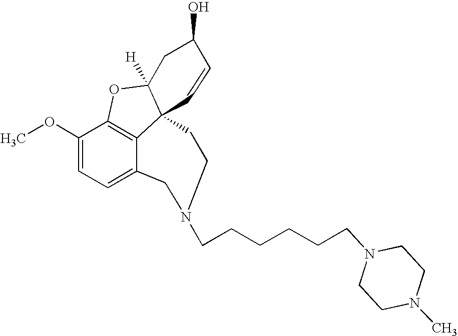 | (−) | | |
| 240 | 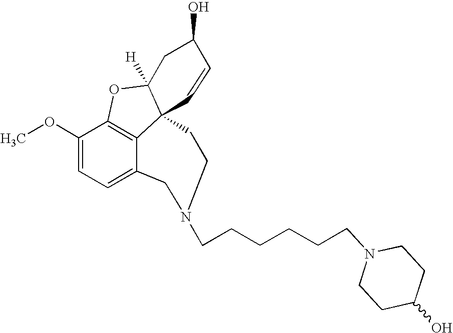 | (−) | | |
| 241 | 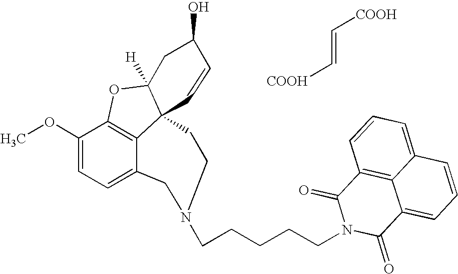 | (−) | | |
| 242 | 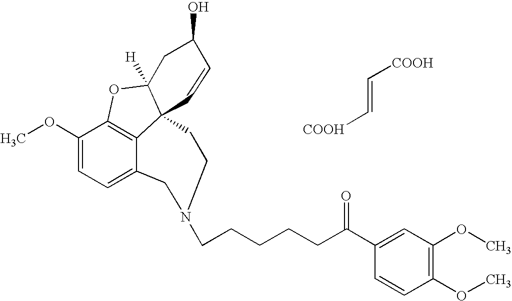 | (−) | | |
| 243 | 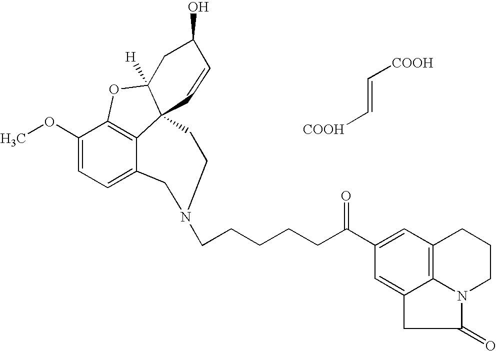 | (−) | | |
| 244 | 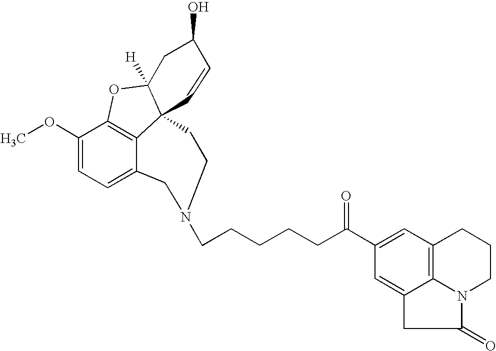 | (−) | | |
| 245 | 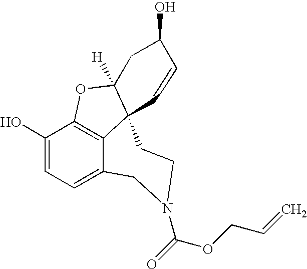 | (−) | | |
| 246 | 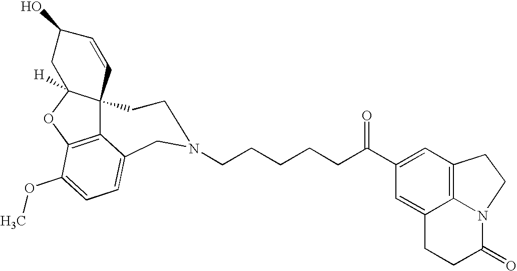 | (−) | | |
| 247 | 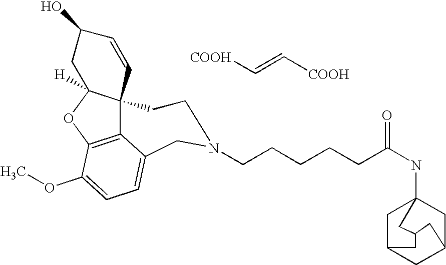 | (−) | | |
| 248 | 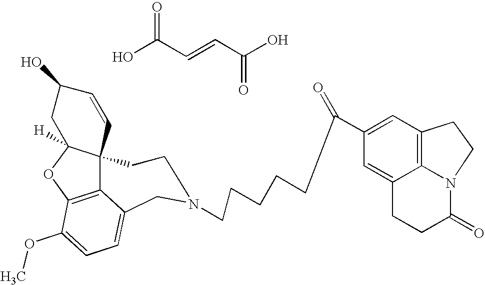 | (−) | | |
| 249 | 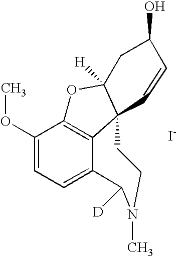 | (−) | | |
| 250 | 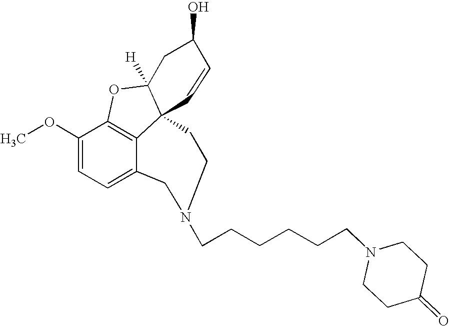 | (−) | | |
| 251 | 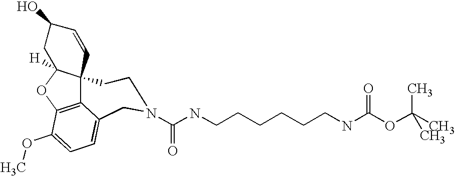 | (−) | | |
| 252 | 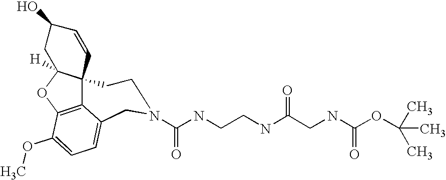 | (−) | | |
| 253 | 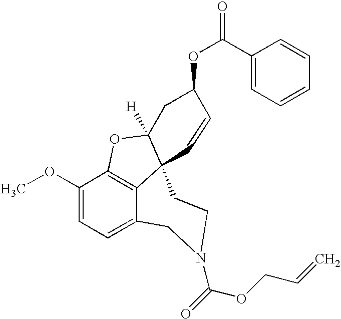 | (−) | | |
| 254 | 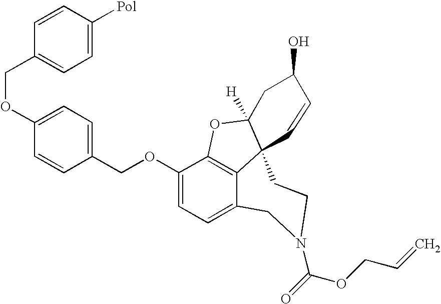 | (−) | | |
| 255 | 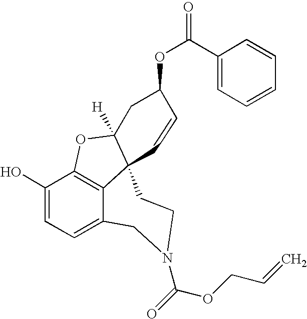 | (−) | | |
| 256 | 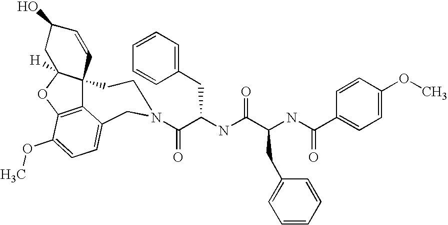 | (−) | | |
| 257 | 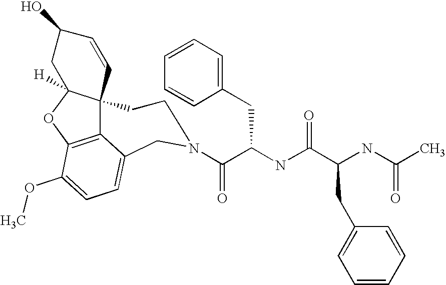 | (−) | | |
| 258 | 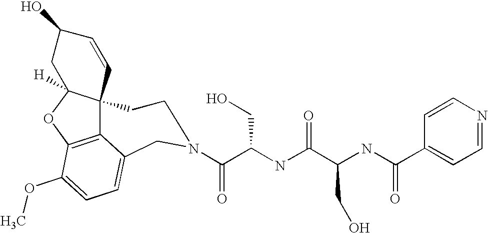 | (−) | | |
| 259 | 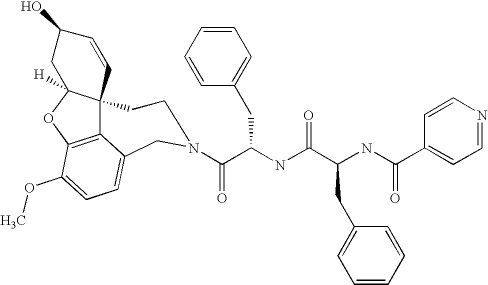 | (−) | | |
| 260 | 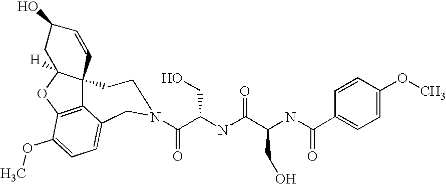 | (−) | | |
| 261 | 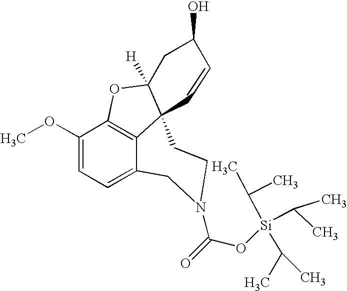 | (−) | | |
| 262 | 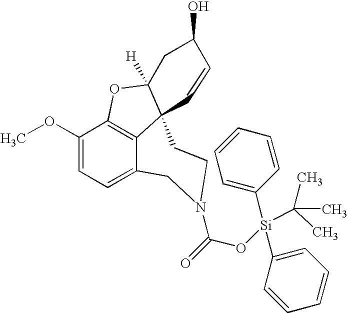 | (−) | | |
| 263 | 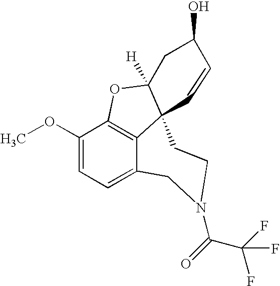 | (−) | | |
| 264 | 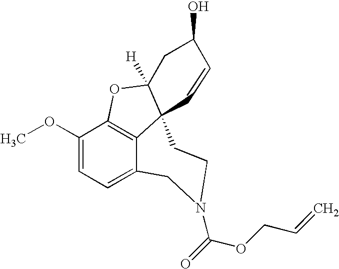 | (−) | | |
| 265 | 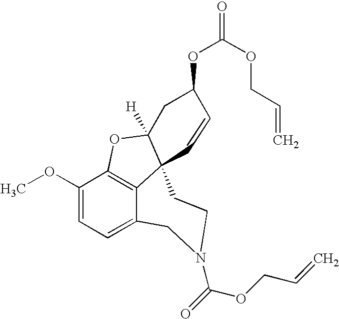 | (−) | | |
| 266 | 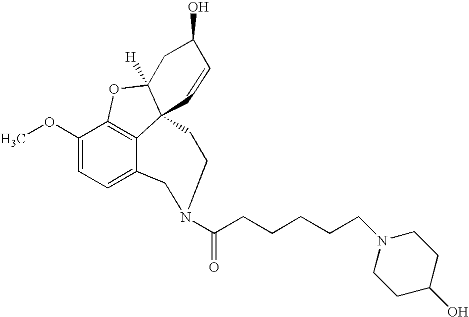 | (−) | | |
| 267 | 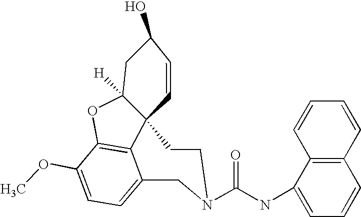 | (−) | 70 | >100 |
| 268 | 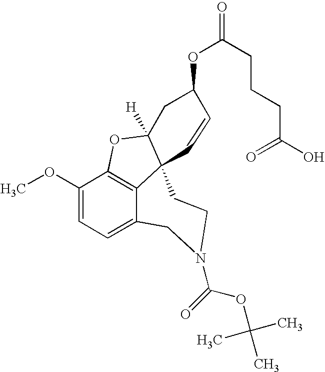 | (−) | | |
| 269 | 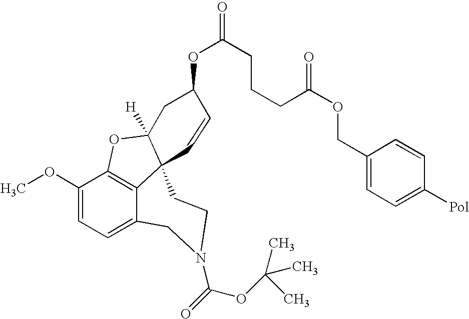 | (−) | | |
| 270 | 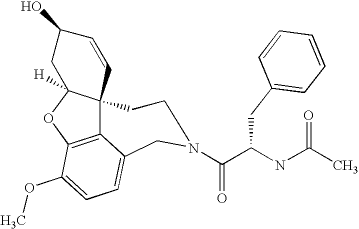 | (−) | | |
| 271 | 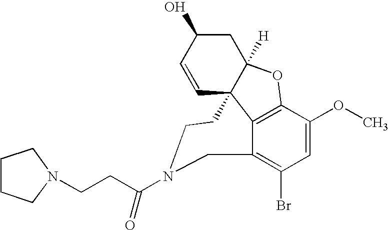 | (+) | >100 | 66 |
| 272 | 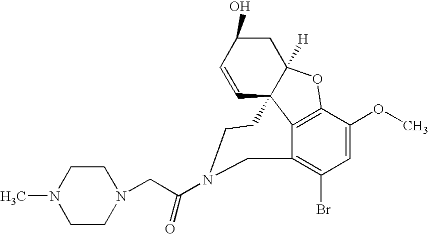 | (+) | 89 | >100 |
| 273 | 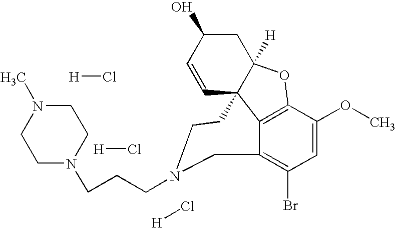 | (+) | >100 | 31 |

According to the measured values shown in the table, there is proof for the cholinergic activity of the compounds of the invention, more precisely for the characteristic inhibition of the cholinergic effect of cholinesterases has been provided and therefore these chemical compounds are used to manufacture medicaments for the treatment as well as for the preventive treatment of post-operative delirium and/or subsyndromes of post-operative delirium.

The galanthamine and its derivatives are used as medicaments containing the active substances or a combination of active substances can also be used. Combinations of the invention are also intended to include combinations with other pharmaceutical active substances.

It has now been determined and confirmed by an extensive clinical study that oral administration of galanthamine (as the hydrobromide under the label name of the Reminyl® and used commercially for the therapy of light to moderately severe Alzheimer's illness) to preoperative patients with limited cognitive ability with acute POD, there was an unexpected and large improvement of the symptoms. As particularly surprising must be the fact that the observed side effects of galanthamine administration were very small, although post-operative patients exhibit a increased cholinergic sensitivity according to the observations.

This is to be described on the basis the following applications examples:

### EXAMPLE 1

The administration of galanthamine or its pharmaceutically acceptable salts and solvates for the therapy or prophylaxis of post-operative deliriums can take place orally (in the form of tablets, capsules, oral solutions or fast-dissolving tablets), intravenously, rectal (in the form of suppositories) or transdermal (in the form of passive or active skin delivering systems of galanthamine).

A preferred form of administration takes place orally, wherein an exemplary administration pattern consists of 8 mg galanthamine hydrobromide given in the form of the active substance directly in free tablets or drinking solutions for the prophylaxis of post-operative deliriums in the evening after the surgical intervention. On the following four days following the operation day in the morning and at noon 4 mg each are given, then in the evening 8 mg are given. On the fifth post-operative day in each case 4 mg are given in the morning and at noon and the prophylaxis is then terminated. It is understood that the specialists can adjust these dosages according to the body weight of the patient, the general state, etc.

Galanthamine hydrobromide-containing tablets with direct release of the active substance are suitable according to the invention for this kind of administration, and are approved under the trade name Reminyl® for the therapy of the Alzheimer's illness.

Galanthamine-containing oral solutions, which are suitable according to invention for this kind of administration, are described in WO-0130318, wherein such an oral solution can be made in exemplary way as follows:

| | | |
|---|---|---|
| Galanthamine HBr | 5.124 | mg |
| Methyl | 1.8 | mg |
| 4-hydroxybenzoate | | |
| Propyl | 0.2 | mg |
| 4-hydroxybenzoate | | |
| Sodium Saccharin di- | 0.5 | mg |
| hydrate | | |
| Water (pH 4.9-5.1) | 1.0 | ml |

A further oral administration pattern uses capsules with retarded release of the active agent, wherein in the evening after the surgical intervention 8 mg galanthamine hydrobromide are given and on the four days following the surgical procedure at noon or in the evening in each case 8 mg are given too. The capsules usable according to the invention having retarded release of the active agent can be made as described in the document WO 0038686, and the entire teachings of the document are further preferred.

Preferred pharmaceutical forms according to invention are transdermal, and the passive transdermal systems described in WO-9416707 are especially suitable. In this case, a transdermal patch, which releases 10 mg free galanthamine in the span of 24 hours, immediately after waking up from the administration of anesthetic and on the next four days replaced by a new patch in each case; and on the fifth day no more renewed application takes place.

Of course, combinations of different modes of administration of the active pharmaceuticals described here are possible. In particular, it proves useful to use daily transdermal administration rather than the faster effect oral administration in the evening of the operation such as by providing an oral dose of 4 mg Galanthamine HBr (directly setting free the active from the tablet or oral solution).

### EXAMPLE 2

### The administration of (4aS,6R,8aS)-6-Hydroxy-3-methoxy-11-methyl-4a,5,9,10-tetrahydro-6H-benzofuro[3a,3,2-ef][2]benzazepinium took place for example with bromide as the counterion. This example concerns a galanthamine derivative with the following structural formula

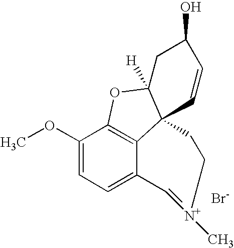 It is however also possible to provide the administration by means of pharmacological acceptable hydrates and solvates. The therapy or prophylaxis of post-operative delirium can take place orally (in the form of tablets, capsules, oral solutions or fast-dissolving tablets), intravenously, rectally (in the form of suppositories) or transdermally (in passive or active form as with the aforementioned skin delivering systems). A preferential form of administration takes place orally, wherein an exemplary administration pattern consists for the prophylaxis of the post-operative delirium that in the evening after the surgical intervention, 2-6 mg of (4aS,6R,8aS)-6-Hydroxy-3-methoxy-11-methyl-4a,5,9,10-tetrahydro-6H-benzofuro[3a,3,2-ef][2]benzazepinium bromide are given as the active substance directly in the form of free tablets or oral solutions. On the four days following the operation day in each case 1-3 mg are then given in the morning and at noon, and in the evening 2-6 mg. On the fifth post-operative day in each case 1-3 mg are given in the morning and at noon and the prophylaxis is then terminated. It is understood that the specialist can automatically adjust these dosages according to the body weight of the patient, their general state, etc. Likewise in place of bromide, there can be used also different physiologically acceptable, easily water-soluble salts of the active substance (e.g. different halide, maleate, tartrate).

(4aS,6R,8aS)-6-Hydroxy-3-methoxy-11-methyl-4a,5,9,10-tetrahydro-6H-benzofuro[3a,3,2-ef][2]benzazepinium bromide containing tablets having direct release of the active substance, are suitable for administration according to the invention, and they can also be provided with pharmaceutical acceptable coatings. For example:

| | | |
|---|---|---|
| (4aS,6R,8aS)-6-Hydroxy-3- | 2.0 | mg |
| methoxy-11-methyl-4a,5,9,10- | | |
| tetrahydro-6H- | | |
| benzofuro[3a,3,2- | | |
| ef][2]benzazepinium bromide | | |
| calcium phosphate | 25.0 | mg |
| Lactose | 5.0 | mg |
| Wheat starch | 5.0 | mg |
| Microcrystalline cellulose | 40 | mg |
| Talc | 2 | mg |
| Magnesium stearate | 1.0 | |

The specialist based on the above examples with application experience in usual pharmaceutical practices specified for galanthamine, can easily make similar pharmaceutical forms for (4aS,6R,8aS)-6-Hydroxy-3-methoxy-11-methyl-4a,5,9,10-tetrahydro-6H-benzofuro[3a,3,2-ef][2]benzazepinium bromide or similar salts, hydrates or solvates.

In order to be able to test the effect of the pharmaceutical forms of the invention on patients, a prospective study was accomplished for the prevention of post-operative delirium at five Austrian orthopedic hospitals (two in Vienna, and one each in Linz, Graz and Krems) and all together 229 patients, who underwent hip replacement and/or combined planned surgical intervention for implantation were part of the study.

Hip/Knee endoprosthesis. The patients of the group were given in the evening following the surgical intervention (day 0) 8 mg galanthamine HCl, then on the days 1 to 4 in each case 4 mg in the morning and at noon and 8 mg in the evening, i.e., 16 mg t.i.d. to the fifth day. The day after the intervention, the dose was reduced to 8 mg b.i.d., starting from that day until the 6th day when no more treatment took place. Patients in the placebo group did not receive distinguishable placebo tablets according to the same pattern.

For the determination of the effectiveness with the help of the “Confusion Assessment Method” (Lit.14) 155 patients could be consulted. In the group of placebos 7 patients (8, 5%) developed an post-operative delirium, in the galanthamine group only one patient (1, 4%) developed post-operative delirium, which corresponds to a statistically significant difference (p=0, 044).

The evaluation of the study shows thus in clear way the effectiveness of galanthamine with post-operative delirium.

### LITERATURE

1. Trzepacz P T. Update on the neuropathogenesis of delirium. Dement Geriatr Cogn Disord. 1999; 10:330-334.
2. Bekker A Y, Weeks E J. Cognitive function after anaesthesia in the elderly. Best Pract Res Clin Anaesthesiol. 2003; 17:259-272.
3. O'Brien D. Acute postoperative delirium: definitions, incidence, recognition, and interventions. J Perianesth Nurs. 2002; 17:384-392.
4. Williams-Russo P, Urquhart B L, Sharrock N E et al. Post-operative delirium: predictors and prognosis in elderly orthopedic patients. J Am Geriatr Soc. 1992; 40:759-767.
5. Kiely D K, Bergmann M A, Murphy K M et al. Delirium among newly admitted postacute facility patients: prevalence, symptoms, and severity. J Gerontol A Biol Sci Med Sci. 2003; 58:M441-M445.
6. Jackson J C, Ely E W. The Confusion Assessment Method (CAM). Int J Geriatr Psychiatry. 2003; 18:557-558.
7. Carnes M, Howell T, Rosenberg M et al. Physicians vary in approaches to the clinical management of delirium. J Am Geriatr Soc. 2003; 51:234-239.
8. Cole M, McCusker J, Dendukuri N et al. The prognostic significance of subsyndromal delirium in elderly medical inpatients. J Am Geriatr Soc. 2003; 51:754-760.
9. Baraka A, Harik S. Reversal of central anticholinergic syndrome by galanthamine. J Am Med Assoc. 1977; 238:2293-2294.
10. Milam S B, Bennett C R. Physostigmine reversal of drug-induced paradoxical excitement. Int J Oral Maxillofac Surg. 1987; 16:190-193.
11. Savage G J, Metzger J T. The prevention of postanesthetic delirium. Plast Reconstr Surg. 1978; 62:81-84.
12. Mulsant B H, Pollock B G, Kirshner M et al. Serum anticholinergic activity in a community-based sample of older adults: relationship with cognitive performance. Arch Gen Psychiatry. 2003; 60:198-203.
13. Santos M D, Alkondon M, Pereira E F et al. The Nicotinic Allosteric Potentiating Ligand Galanthamine Facilitates Synaptic Transmission in the Mammalian Central Nervous System. Mol Pharmacol. 2002; 61:1222-1234.
14. Inoue S. K., van Dyck C. H., Alessi C. A. et al.: Clarifying confusion: the confusion assessment method. A new method for the detection of delirium. Ann Intern Med. 1990; 113 (12):941-8

1. (canceled)

2. (canceled)

3. A method according to claim 14, wherein the galanthamine derivatives have the general formula 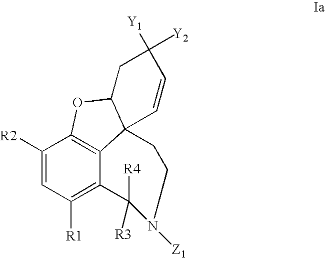
and the salts thereof, wherein R₁ is H, branched or straight chain (C₁-C₆) alkyl, Br, NO₂, NR₅R₆ wherein R₅ and R₆ are the same or different and are selected from H, branched or straight chain (C₁-C₆) alkyl, and wherein R₂ is OH, branched or straight chain (C₁-C₆) alkyl, methoxy, phenyloxy or the following group 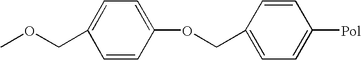
whereby Pol is a polymer, and wherein R₃ and R₄ either at the same time or alternatively are H, D, CN, straight chain or branched (C₁-C₆) alkyl or a carbonyl group together, wherein Y₁ and Y₂ alternatively are H or a group selected from: 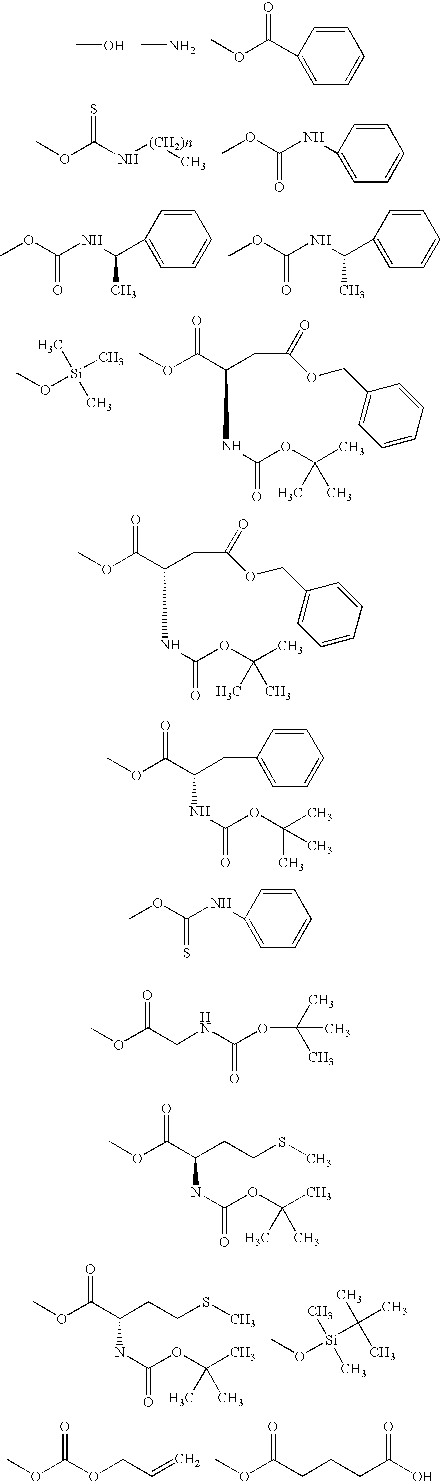 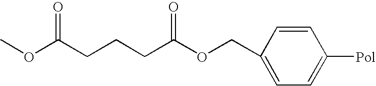
wherein n represents a value of 0, 1 to 15, and Pol has the meaning indicated above, and wherein Y₁ and Y₂ further represent together a carbonyl group (═O), ═NH, ═N—OR₇, wherein R₇ is H, tosylate or branched or straight chain (C₁-C₆) alkyl, or Y₁ and Y₂ together is a group selected from: 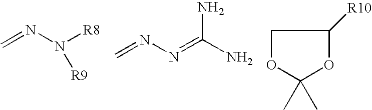
wherein R₈ and R₉ are the same or different and are H, branched or straight chain (C₁-C₆) alkyl, —(CH₂)₂—OH, CHO, CONH₂, tBOC (tert-Butoxycarbonyl), or mean —COCOOH, R₁₀ is H or CH₃, and wherein when Y, is —O—(CH₂)₂—OH, Y₂ is OH, and wherein Z₁ is H, branched or straight chain (C₁-C₆) alkyl, (C₂-C₇) alkenyl, (C₂-C₇) alkynyl, tri-fluoroacetyl, formyl, phenyl or a group selected from: 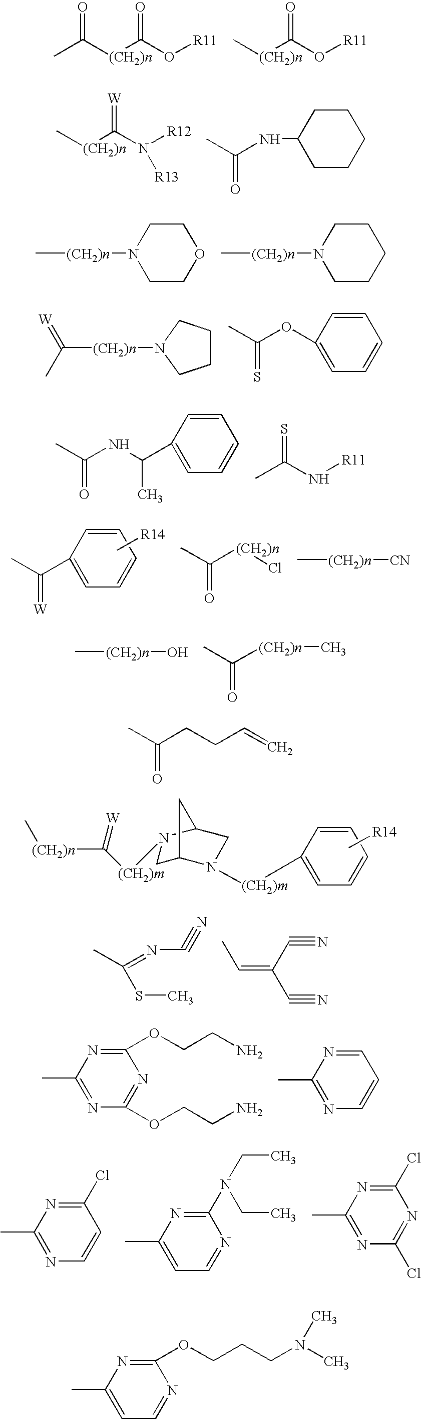 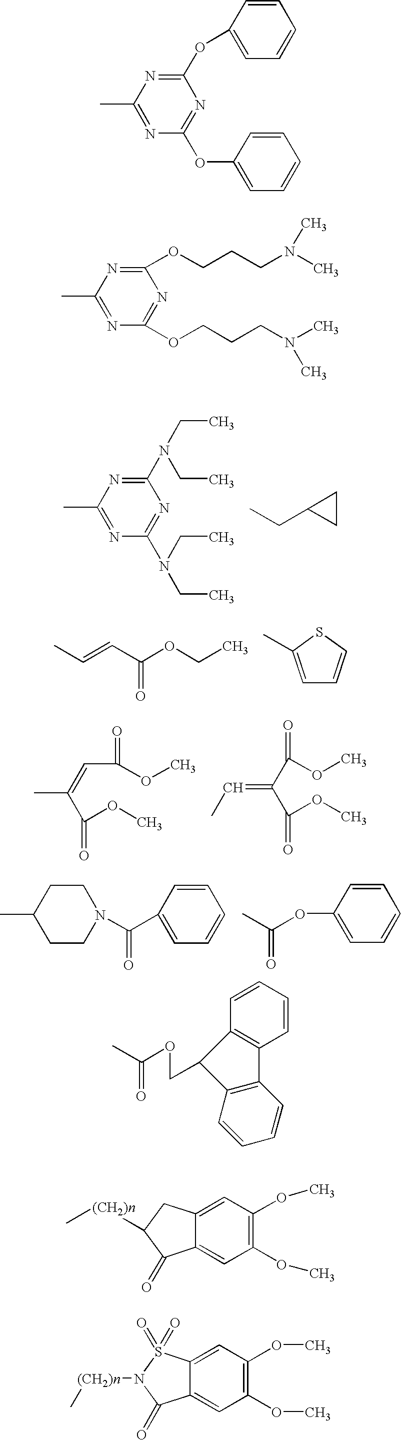 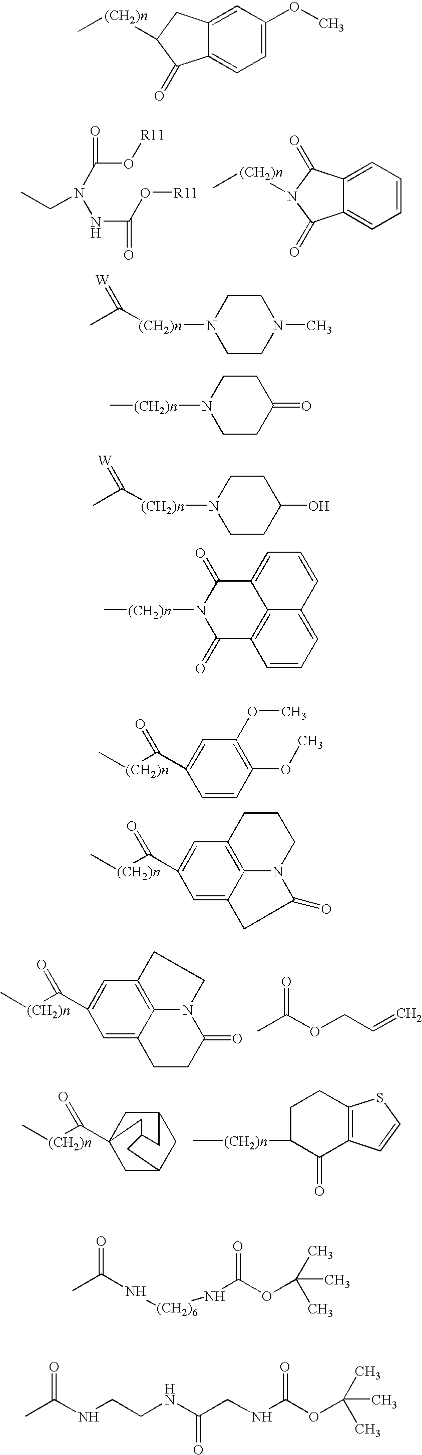 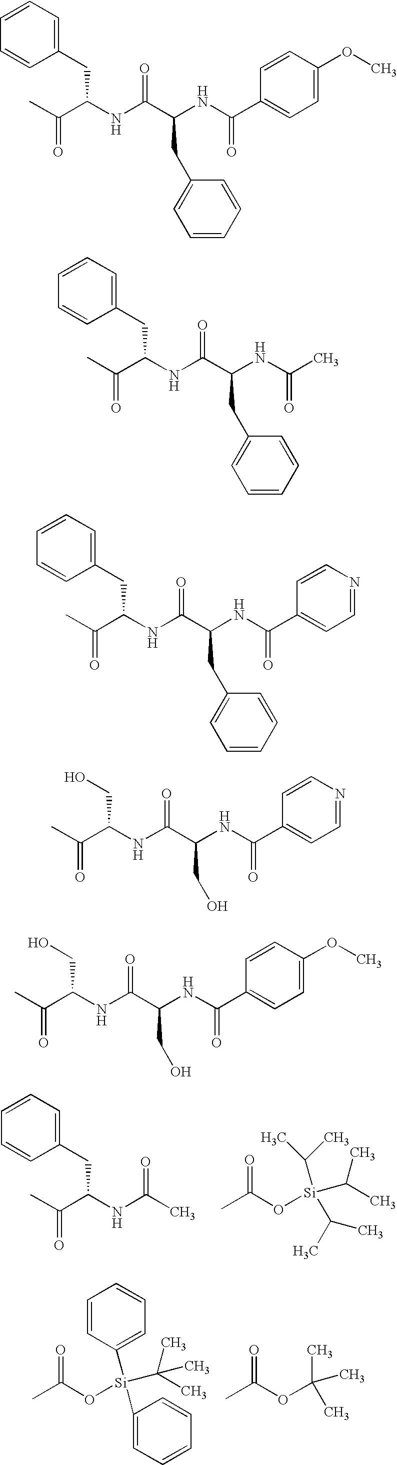
wherein R₁₁ is H, straight chain (C₁-C₆) alkyl, branched (C₁-C₆) alkyl or (C₂-C₇) alkenyl, R₁₂ and R₁₃ are the same or different and are selected from H, straight chain or branched (C₁-C₆) alkyl, phenyl, chlorophenyl, (trifluoromethyl)-phenyl or 1-naphtyl, wherein R₁₄ is H, F, CH₃, NO₂, Cl, Br, J, CF₃, n has the meaning indicated above, m is 0 or 1, and W has the meaning H or O, and wherein further Z₁ and R₃ form a common ring 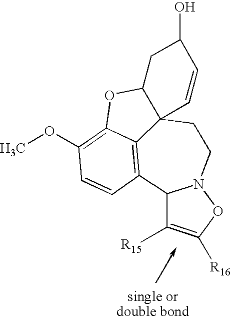
wherein R₁₅ and R₁₆ alternatively mean H, COOCH₃, COOCH₂CH₃, CN, COCH₃.

4. A method according to claim 14, wherein the galanthamine derivatives have the general formula Ib 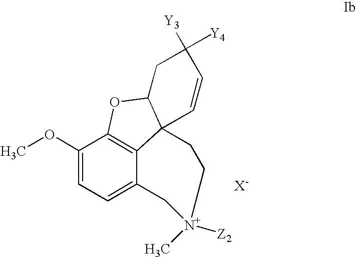
wherein Y₃ and Y₄ alternatively mean H and OH, X is Cl, Br or I, Z₂ is oxygen (N-oxide and no counterion), branched or straight chain (C₁-C₆) alkyl, or (C₂-C₇) alkenyl or (C₂-C₇) alkynyl or a group selected from: 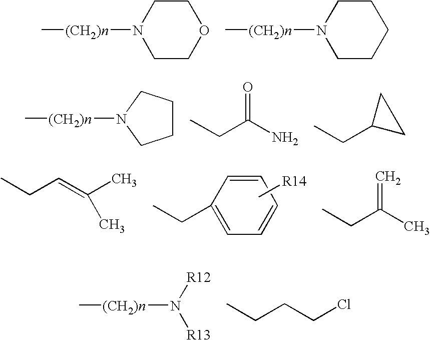
wherein R₁₂ and R₁₃ are the same or different and are selected from H, straight chain or branched (C₁-C₆) alkyl, phenyl, chlorophenyl, (trifluoromethyl)-phenyl or 1-naphtyl, wherein R₁₄ is H, F, CH₃, NO₂, Cl, Br, J, CF₃, and n has the meaning indicated above.

5. A method according to claim 14, wherein the galanthamine derivatives have the general formula Ic 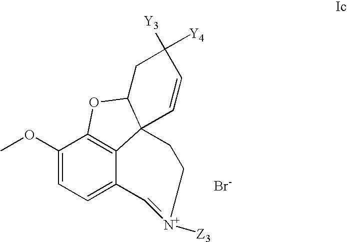
wherein Y₃ and Y₄ alternatively are H or OH, and Z₃ is oxygen (N-oxide and no counterion) or is a methyl.

6. A method according to claim 14, wherein the galanthamine derivatives have the general formula Id 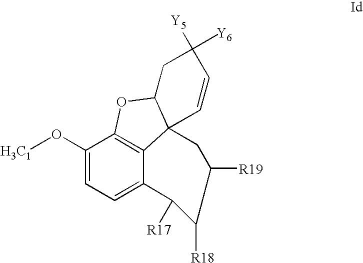
and their salts, wherein Y₅ and Y₆ alternatively are H or OH, or together form a keto group, and R₁₇, R₁₈, R₁₉ are alternatively for two substituents H, wherein the third substituent is NH₂ or CONH₂.

7. A method according to claim 14, wherein the galanthamine derivatives have the general formula Ie 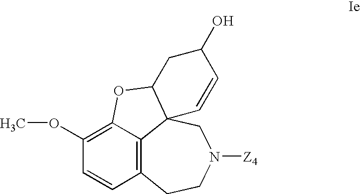
or their salts, wherein Z₄ is straight chain or branched (C₁-C₆) alkyl or 4-brombenzyl.

8. A method according to claim 14, wherein the galanthamine derivatives have the general formula If: 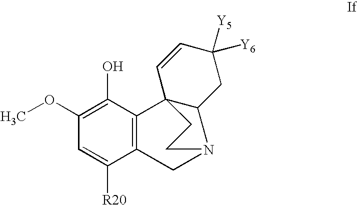
or their salts, wherein Y₅ and Y₆ alternatively are H or OH, and R₂₀ is H or Br.

9. A method according to claim 14, wherein the galanthamine derivative has the following structural formula 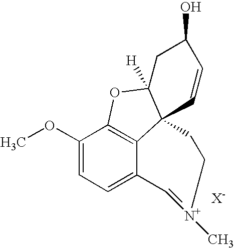
and its pharmaceutical acceptable salts, hydrate or a solvate thereof and having the chemical name (4aS,6R,8aS)-6-Hydroxy-3-methoxy-11-methyl-4a,5,9,10-tetrahydro-6H-benzofuro[3a,3,2-f][2]benzazepinium.

10. A method according to claim 9, wherein the pharmaceutical acceptable salt counterion of (4aS,6R,8aS)-6-Hydroxy-3-methoxy-11-methyl-4a,5,9,10-tetrahydro-6H-benzofuro[3a,3,2-ef][2]benzazepinium is selected from the group of halides, carboxylic acids with 1-3 carboxyl functions, sulfonic acids.

11. A method according to claim 10, wherein the counterion is bromide.

12. A method according to claim 10, wherein the counterion is selected from the group consisting of tartrate, malonate, fumarate and succinate.

13. A method according to claim 10, wherein the counterion is methane sulfonic acid.

14. A method of treating post-operative delirium or subsyndromes of post-operative delirium in a patient, comprising:
administering to the patient an effective amount of a compound selected from the group consisting of galanthamine and galanthamine derivatives exhibiting cholinergic activity.
